SEQUENCE LISTING (44 sequences)

SEQ ID NO: 1 - DNA, 1119 bp, Artificial Sequence
  Synthetic Construct
atgaaaatcggcatcccgaaagaaatcaaaaacaacgaaaaccgcgtcgcgatcacgccggcgggcgtcatgacgctggtcaaagcgggccatgacgtctatgtcgaaacggaagcgggcgcgggcagcggctttagcgacagcgaatatgaaaaagcgggcgcggtcatcgtcccgaacgcggaagacgcgtggacggcggaaatggtcctgaaagtcaaagaaccgctggcggaagaatttcgctattttcgcccgggcctgatcctgtttacgtatctgcatctggcggcggcggaagcgctgacgaaagcgctggtcgaacagaaagtcgtcggcatcgcgtatgaaacggtccagctggcgaacggcagcctgccgctgctgacgccgatgagcgaagtcgcgggccgcatgagcgtccaggtcggcgcgcagtttctggaaaaaccgcatggcggcaaaggcatcctgctgggcggcgtcccgggcgtccgccgcggcaaagtcacgatcatcggcggcggcacggcgggcacgaacgcggcgaaaatcgcggtcggcctgggcagcgacgtcacgatcctggacatcaacgcggaacgcctgcgcgaactggacgacctgtttggcgaccatgtcacgacgctgatgagcaacagctatcatatcgcggaatgcgtccgcgaaagcgacctggtcgtcggcgcggtcctgatcccgggcgcgaaagcgccgaaactggtcacggaagaaatggtccgcagcatgacgccgggcagcgtcctggtcgacatcgcgatcgaccagggcggcatctttgaaacgacggaccgcgtcacgacgcatgacgacccgacgtatgtcaaacatggcgtcgtccattatgcggtcgcgaacatgccgggcgcggtcccgcgcacgagcacgtttgcgctgacgaacgtcacgatcccgtatgcgctgcagatcgcgaacaaaggctatcgcgcggcgtgcctggacaacccggcgctgctgaaaggcatcaacacgctggacggccatatcgtctatgaagcggtcgcggcggcgcataacatgccgtatacggacgtccatagcctgctgcatggctaa

SEQ ID NO: 2 - DNA, 1173 bp, Bacillus licheniformis
atgatgagcttaaaaccattctatagaaagacatgggccgaaatcgatttaacggctttaaaagaaaacgtccgcaatatgaagcggcacatcggcgagcatgtccgcctgatggccgtcgttaaagcgaatgcctacggacacggggatgcacaggtagcgaaggcggctcttgcagaaggggcgtccattcttgctgtggctttattggatgaagcgctttcgctgagggcgcaggggattgaagaaccgattcttgtcctcggtgcagtgccgaccgaatatgcaagcattgccgcggaaaagcgcattatcgtgactggctactccgtcggctggctgaaagacgtgctcggttttctgaatgaggccgaagctcctcttgaatatcatttgaagatcgacacgggcatgggccgccttggctgcaaaacggaagaagagatcaaagaaatgatggagatgaccgaatcgaacgataagctcaattgtacgggcgtgttcactcatttcgccacggcggacgaaaaggacaccgattatttcaacatgcagcttgaccgctttaaagagctgatcagccccctcccgcttgaccgtttgatggtgcattcgtcaaacagcgccgcgggtctgcgcttcagggaacagctatttaatgccgtccgcttcggcatcggcatgtacggtttggcgccgtcaaccgaaataaaagacgagctgccgtttcgtctgcgggaagtgttttcgcttcataccgaactcacccatgtgaaaaaaattaaaaaaggcgagagcgtcagctacggggcgacatatacagctcagcgcgacgaatggatcgggacagtccccgtcgggtatgccgacggatggctgaggcgcctggccggaacggaagtgctgatcgacggaaaacgccaaaaaatagcagggagaatctgcatggaccagttcatgatttcccttgccgaagaataccctgtcggcacaaaggttaccttgatcggaaagcaaaaagacgaatggatctcagtcgacgaaatcgcccaaaatttgcagacgatcaattatgaaattacctgtatgataagttcaagggtgccccgtatgtttttggaaaatgggagtataatggaaataaggaatccgatcttgcctgatcaatcctga

SEQ ID NO: 3 - DNA, 1182 bp, Bacillus licheniformis
atgaaaaagctttgccgtgaagtttgggtagaggtaaatcttgatgcgatcaaaaaaaatttgcgcgcgtttcggcggcatattccgaaaaagagcaaaattatggctgtcgtaaaagcgaatgcttatggtcacggatcggtggaagttgcacgccatgcacttgaacatggtgcgagtgagctcgccgttgcctcggtggaggaagcggtcgttttacgaaaagcggggattaaagcgccgatccttgtgcttggtttcaccccgctgagctgtgtgaaagaagcggcagcttggaatatatcgttatcagcttttcaagttgactggattaaagaagcgaacgagatattggaaaatgaagcagatcctaaccggctggctgttcatatcaatgtggataccggcatggggcgtttaggtgtacgaacaaaggaaaagcttttagcaatcgtggaagcgctgacggcaagtgaaaacctcgaatgggaaggaatttttacgcatttttccacagctgacgaaccggatactgagctaaccatgattcaacacgaaaagtttatcagctttcttcgctttctgaaagaacaaggctttaagctgcctacggtgcatatgaacaatacggccgcggcgatcgcttttccggaattcagcgctgatatgattcgcttaggcatcggaatgtatggattatatccttccgattatatcaggcagcttaatctcgttaagcttgtgcctgcactaagcttgaaggcgcgaatcgcttatgtgaaaaccatgttgactgaaccgcggacggttagttatggtgctacatatgttgcagagcgcggggaagtcattgccacaattccggtcggctatgctgacggctattcccgtgaactttccaaccgcggttttattcttcatcgaggaagacgagtgccggtggcgggaagagtaacaatggatatgataatggtcagtctgggagagggtgaaggtaaacaaggagaggaagtcgtgatttacggccggcaaaagggagcagagatatctgttgatgaaattgcggaaatgcttgatacgatcaactatgaagtggtatctaccataagctggcgcgtccctcgtttttatataagagacggcgagatttttaaaaagtcgaccccgctgttatacgtgtag

SEQ ID NO: 4 - DNA, 300 bp, Artificial Sequence
  Synthetic Construct
aaggtgacgcctatttcactttctagctgtttaatctgctggctgagcggaggctgagtcatgttcagccgaagagctgcttttccgaaatgcagttcttcggcaacaaccataaaataacgaagatggcgcagctccattaatcactcattcctttctgaatgcgatttcagtcgttttacatattaattgtaagacaaagaagtattggaaaacaatttccacaagatgtatatttaataatacaataattttattaaaaattcattgtaaatgaatgaaaatggaggagtgagggct

SEQ ID NO: 5 - DNA, 48 bp, Artificial Sequence
  Synthetic Construct
cctcgcgtcgggcgatatcggatccgaaggggaaagtcttcgatttct

SEQ ID NO: 6 - DNA, 37 bp, Artificial Sequence
  Synthetic Construct
agagggctttttcatgctgaagaggtcaaaaagagcc

SEQ ID NO: 7 - DNA, 38 bp, Artificial Sequence
  Synthetic Construct
tttgacctcttcagcatgaaaaagccctctttgaaaag

SEQ ID NO: 8 - DNA, 47 bp, Artificial Sequence
  Synthetic Construct
ccatggtacccgggagctcgaattccataagaccgctgatgacaagc

SEQ ID NO: 9 - DNA, 47 bp, Artificial Sequence
  Synthetic Construct
tccagcctcgcgtcgggcgatatcgtccccataacaacggaatcatc

SEQ ID NO: 10 - DNA, 37 bp, Artificial Sequence
  Synthetic Construct
aataggcgtcaccttgactcatcattcctttgccgtt

SEQ ID NO: 11 - DNA, 38 bp, Artificial Sequence
  Synthetic Construct
aaggaatgatgagtcaaggtgacgcctatttcactttc

SEQ ID NO: 12 - DNA, 36 bp, Artificial Sequence
  Synthetic Construct
tccaattcgcttcatagccctcactcctccattttc

SEQ ID NO: 13 - DNA, 39 bp, Artificial Sequence
  Synthetic Construct
ggaggagtgagggctatgaagcgaattggagtattgaca

SEQ ID NO: 14 - DNA, 36 bp, Artificial Sequence
  Synthetic Construct
cttcatggtgttcagttacaatacagtcgcatggcc

SEQ ID NO: 15 - DNA, 42 bp, Artificial Sequence
  Synthetic Construct
gcgactgtattgtaactgaacaccatgaagatactaacatca

SEQ ID NO: 16 - DNA, 48 bp, Artificial Sequence
  Synthetic Construct
actagacagatctatcgatgcatgctttcccttattcctttaaacccg

SEQ ID NO: 17 - DNA, 37 bp, Artificial Sequence
  Synthetic Construct
aataggcgtcaccttgctgaagaggtcaaaaagagcc

SEQ ID NO: 18 - DNA, 39 bp, Artificial Sequence
  Synthetic Construct
tttgacctcttcagcaaggtgacgcctatttcactttct

SEQ ID NO: 19 - DNA, 36 bp, Artificial Sequence
  Synthetic Construct
agagggctttttcatttagccatgcagcaggctatg

SEQ ID NO: 20 - DNA, 38 bp, Artificial Sequence
  Synthetic Construct
ctgctgcatggctaaatgaaaaagccctctttgaaaag

SEQ ID NO: 21 - DNA, 50 bp, Artificial Sequence
  Synthetic Construct
cctcgcgtcgggcgatatcggatccaaaatatgacgctgtctcaaattga

SEQ ID NO: 22 - DNA, 40 bp, Artificial Sequence
  Synthetic Construct
ctaattcatcaatttgacacttcctgttccttgtttcact

SEQ ID NO: 23 - DNA, 39 bp, Artificial Sequence
  Synthetic Construct
ggaacaggaagtgtcaaattgatgaattagcggaaaaac

SEQ ID NO: 24 - DNA, 48 bp, Artificial Sequence
  Synthetic Construct
ccatggtacccgggagctcgaattccggagtctctttcaaaaccgtag

SEQ ID NO: 25 - DNA, 47 bp, Artificial Sequence
  Synthetic Construct
cctcgcgtcgggcgatatcggatccaaaatcatgtaagcccattccg

SEQ ID NO: 26 - DNA, 37 bp, Artificial Sequence
  Synthetic Construct
gtgagtatgggaaaacaacgctcccttctttcttgtc

SEQ ID NO: 27 - DNA, 36 bp, Artificial Sequence
  Synthetic Construct
aagaagggagcgttgttttcccatactcacaggccg

SEQ ID NO: 28 - DNA, 47 bp, Artificial Sequence
  Synthetic Construct
ccatggtacccgggagctcgaattctaaaatgaaggtggtccgggat

SEQ ID NO: 29 - DNA, 47 bp, Artificial Sequence
  Synthetic Construct
cctcgcgtcgggcgatatcggatccaagtgaaaggcgaagagcaaac

SEQ ID NO: 30 - DNA, 37 bp, Artificial Sequence
  Synthetic Construct
aataggcgtcaccttttattttacaacgtggatcggc

SEQ ID NO: 31 - DNA, 39 bp, Artificial Sequence
  Synthetic Construct
cacgttgtaaaataaaaggtgacgcctatttcactttct

SEQ ID NO: 32 - DNA, 36 bp, Artificial Sequence
  Synthetic Construct
ttttaagctcatcatagccctcactcctccattttc

SEQ ID NO: 33 - DNA, 43 bp, Artificial Sequence
  Synthetic Construct
ggaggagtgagggctatgatgagcttaaaaccattctatagaa

SEQ ID NO: 34 - DNA, 37 bp, Artificial Sequence
  Synthetic Construct
gagggggtttttgattcaggattgatcaggcaagatc

SEQ ID NO: 35 - DNA, 35 bp, Artificial Sequence
  Synthetic Construct
cctgatcaatcctgaatcaaaaaccccctctgcag

SEQ ID NO: 36 - DNA, 42 bp, Artificial Sequence
  Synthetic Construct
ccatggtacccgggagctcgaattcatattattgtgccgaac

SEQ ID NO: 37 - DNA, 36 bp, Artificial Sequence
  Synthetic Construct
gcaaagctttttcatagccctcactcctccattttc

SEQ ID NO: 38 - DNA, 36 bp, Artificial Sequence
  Synthetic Construct
ggaggagtgagggctatgaaaaagctttgccgtgaa

SEQ ID NO: 39 - DNA, 37 bp, Artificial Sequence
  Synthetic Construct
gagggggtttttgatctacacgtataacagcggggtc

SEQ ID NO: 40 - DNA, 35 bp, Artificial Sequence
  Synthetic Construct
ctgttatacgtgtagatcaaaaaccccctctgcag

SEQ ID NO: 41 - DNA, 963 bp, Bacillus coagulans
atgaagcgaattggagtattgacaagcggcggcgatgcaccggggatgaatgcggcggtccgcgcgattgcccgtaaagggatttatcacggcctggaagtttacggcattcgccaaggttataacggattgattcaaggaaacatccaaaagctcgaagcaggatctgttggcgatattctccagcggggcggcacggttttgcagtcggcaagaagcgaagaattcaaaacgccggaagggcagcaaaaagcgatcaggcagctgaaagaccatggcattgaagcgctcgttgtgatcggcggcgacggttcctaccaaggggccaaaaagttgacggaacagggctttaactgcattggtgtgccagggacaatcgataacgacatcccggggacggattttacaatcggttttgatacggcattgaacacagtgcttgatgcgattgataaaattcgcgacaccgcttcttcccacgaacgcacctttattattgaagtcatgggcagaaatgccggggatatcgcgctctggtccggcctggccggcggagccgaatcgattattattccggaagaaaaatatgacttaaaagatgtcgtggagcgtcttgaacaggggagaaaacgcggcaaacgccacagcatcatcattgtcgcggaaggcgtgatgagcggcaacgagtttgctgaacaattgaaaaaaaccggtgtgatcggcgatacccgcgtttctgttctcggccatatccagcgcggcggttctccgacggcatttgaccgcgtgcttgcaagccgcctcggcgcaagggctgttgaactgctgcttgaaggaaaagggggccgcgctgtcggcattcaaaataaccagctggttgaccacgatatccttgagattctcggaaaaccgcacgccgttaataaaaacatgtacaagctgtcgaaagaattgtcgatctaa

SEQ ID NO: 42 - DNA, 1761 bp, Bacillus coagulans
atgaaaaaaaccaaaattgtatgtacaatcggacctgccagtgaaagtgtggaaatgcttgaaagattaatggcaaacgggatggatgtttgccgcctgaacttctcgcacggcagccatgaggaacatcttgcccggattaaaaatatccgtgaagctgcaaaaaaccaaaacaaaacgatcgggcttctgctcgatacaaagggcccggaaatccgcacccatgatatgaaagacggcggattcgagctcgttgaaggcatgacaccggtcatttcaatgacagaagtgctcgggacaccggaaaaattttcggtcacatatgaagggctgattgatgatgtgcacgttggctctaaaattttacttgatgacggtttgattgaactggaagtgacggccatcgataaaaacgccggtgaaatccatacaaaagtgctgaaccgcggcgttttgaaaaacaaaaaaggtgttaacgtcccgggtgtttccgtgaaccttccgggcatcaccgaaaaagacgtgagcgatatcctgttcgggcttgaacaaggcattgacttcattgcggcttcgtttgtacgccggccgtccgacgttttggaaatccgccagctccttgaagaacacgatgctttgcatgtgaaaattttccctaaaattgaaaaccaggaaggcgtcgacaatatcgatgaaatccttgcggtatcagacggcttaatggttgcccgcggcgacctcggcgttgaaattccgaccgaagcggtgccgctcgtacaaaaagaaatgatcagaaaatgtaatacgctcggcaaaccggtgattaccgcaacgcaaatgcttgattcgatgcaacgcaacccgcgcccgacccgcgcggaagcaagcgacgtggccaacgccatttttgacggcacggatgcgatcatgctttccggcgaaacggcagccgggaaatatcctgctgaagcggttaagacgatgtacaatattgcggttcatgtggaaaaagcaattaaccatcgcgatattctgaacaagcgcagcaagagcacggaccataatatgacagacgctatctgccagtccgttgcccatacggctttaaatcttgatgtgaatgccattattgcgccgactgaaagcggctatacggcacgcatgatctccaaataccgcccggcggccccaatcattgctgtcacgagcgatccgaaagtacaacgcggcttaactgttgtgtccggcgtatacccacaattgggcacaaaggcaaacaatacggatgaaatgcttgaaattgcagtggaggaagcgttgaaatccgaaatcgtccatcacggcgaccttgtgatcattacagcaggcgtcccggttggtgggaaaggcaccaccaacctgatgaaagtgcacctgatcggtgatatattggcaaaaggccagggaatcggcagaaaatcggcattcggcccggtcatcgttgctgaaagccctgaagaagcaaacgcaaaggcaacagaaggttgtgtgctcgtcacgagaacgaccgacaaagaaatcatgccggccattgaaaaatgcgccgcgctgattacggaagaaggcggcttgacaagccatgctgcagttgttggcatcaatgtcggcattccggtcattgttggcgttgaaaaagccgtttccatttttgaagacgggcaggaagttacggtagatgcggcaaccggctcggtttacaacggccatgcgactgtattgtaa

SEQ ID NO: 43 - DNA, 978 bp, Bacillus licheniformis
atgaaagtaatttttttttctatgcacccgtatgaagaggaatttctgggtccgattctgccgtctgactgggacgtagaaatgaccccggactttctggacgaaaccaccgtggaaaaggctaaaggtgcccaggtagtaagcctgtttgtttctgacaaagctgatggtccggtactggaagcgctgcattcttacggtgtgggcctgctggccctgcgttctgctggctatgatcacatcgatattgagaccgcaaaacgcctgggtatcaaagtagttaacgtgccagcctattctccgcacgctatcgctgaccatactctggctatcatgctggctctgattcgtcgtctgcaccgtgcccatgataaagtgcgcctgggtgattttgatctggatggtctgatgggctttgatctgaacggcaaagttgctggtgtaattggtctgggtaaaatcggtcgcctggtagctacccgcctgaaagcgtttggttgcaaagttctgggctatgatccatacattcagccggaaatcgtagaaaacgttgatctggataccctgatcactcaggctgatatcatttctattcattgtccgctgacccgtgaaaactttcatatgtttaacgaagagacttttaagcgtatgaaaccgggtgctattctggttaacaccgcgcgtggtggtctgatcgataccaaggccctgctggaggccctgaagtctggtaaactgggcggcgcagccctggatgtgtatgaatatgaacgtggcctgttttttaaaaaccaccaaaaagaaggtatcaaagacccgtatctggcccagctgctgggtctggccaacgtagtgctgaccggtcatcaggcctttctgacccgtgaggctgtaaaaaacatcgaagaaactaccgtagaaaacattctggaatggcaaaagaacccgcaggcaaagctgaaaaacgaaatctaa

SEQ ID NO: 44 - protein, 372 aa, Artificial Sequence
  Synthetic Construct
MKIGIPKEIKNNENRVAITPAGVMTLVKAGHDVYVETEAGAGSGFSDSEYEKAGAVIVPNAEDAWTAEMVLKVKEPLAEEFRYFRPGLILFTYLHLAAAEALTKALVEQKVVGIAYETVQLANGSLPLLTPMSEVAGRMSVQVGAQFLEKPHGGKGILLGGVPGVRRGKVTIIGGGTAGTNAAKIAVGLGSDVTILDINAERLRELDDLFGDHVTTLMSNSYHIAECVRESDLVVGAVLIPGAKAPKLVTEEMVRSMTPGSVLVDIAIDQGGIFETTDRVTTHDDPTYVKHGVVHYAVANMPGAVPRTSTFALTNVTIPYALQIANKGYRAACLDNPALLKGINTLDGHIVYEAVAAAHNMPYTDVHSLLHG

(19) United States
(12) Patent Application Publication
(10) Pub. No.: US 2025/0277225 A1
(43) Pub. Date: Sep. 4, 2025

(54) DL-Alanine-Producing Genetically Engineered Strain and Method of Construction and Use Thereof

(71) Applicant: Anhui BBCA Biochemical Co., Ltd., Bengbu City, Anhui Province (CN)

(72) Inventors: Fei TAO, Shanghai (CN); Xiao HAN, Shanghai (CN); Ping XU, Shanghai (CN); Xiaoling MU, Bengbu City (CN); Weili LI, Bengbu City (CN); Sihong CHEN, Bengbu City (CN)

(21) Appl. No.: 18/698,852

(22) PCT Filed: Dec. 6, 2021
(86) PCT No.: PCT/CN2021/135645

§ 371 Date: Apr. 5, 2024

(30) Foreign Application Priority Data
Oct. 8, 2021 (CN) 202111170973.6

(51) Int. Cl. C12N 15/75; C12N 9/04; C12N 9/06; C12N 9/12; C12N 9/90; C12P 13/06; C12R 1/10

(52) U.S. Cl. CPC C12N 15/75; C12N 9/0006; C12N 9/0016; C12N 9/1205; C12N 9/90; C12P 13/06; C12R 2001/10; C12Y 101/01028; C12Y 104/01001; C12Y 207/01011; C12Y 207/0104; C12Y 501/01001

(57) ABSTRACT

The present invention discloses a DL-alanine-producing genetically engineered strain, as well as a method of construction and use thereof, and pertains to the field of bioengineering. According to the present invention, through enhancing the glycolysis pathway or/and introducing thermostable alanine dehydrogenase, a genetically engineered strain capable of high-yield production of alanine at 42° C. to 55° C. This strain can be used in a two-step method for producing racemic DL-alanine, which includes fermentation and subsequent addition of microbial alanine racemase. Through inactivating or deleting alanine racemase genes in this strain and then separately introducing overexpressed alanine racemase gene(s), a genetically engineered strain capable of producing racemic DL-alanine using a direct fermentation method can be constructed. When the original strain possesses a lactate synthesis pathway, blocking this lactate synthesis pathway in both the genetically engineered strains can additionally augment the proportion of a pyruvate synthesis pathway.

### FIELD OF THE INVENTION

The present invention relates to the field of bioengineering and, in particular, to a DL-alanine-producing genetically engineered strain, as well as to a method of construction and use thereof.

### DESCRIPTION OF THE PRIOR ART

As an important amino acid compound and one of the smallest chiral molecules, alanine exists in three optically active isomeric forms: L-alanine, D-alanine and racemic DL-alanine. Racemic DL-alanine is an important industrial compound widely used in food, medicine, agriculture and other fields. As DL-alanine is an amino acid with strong sweetness, it can be used as a seasoning and nutritional supplement for enhancing the sensory properties of chemical flavorings. In the field of medicine, DL-alanine is a main raw material for producing aminopropanol and vitamin B6, which are raw materials for the synthesis ofloxacin. In the field of agriculture, DL-alanine can be used as a feed additive and in the synthesis of amino acid pesticides.

Production methods of DL-alanine mainly include chemical synthesis, enzymatic catalysis and fermentation. Chemical synthesis includes Strecker synthesis, Buchere synthesis, ammoniation of α-halogenated carboxylic acids, phase-transfer catalytic synthesis and chemical racemization of L-alanine. The first four all suffer from poor safety, difficult isolation, low yield and other problems, while chemical racemization of L-alanine is associated with issues including expensive raw materials, easy corrosion of equipment and environmental pollution and is therefore gradually faded out. Enzymatic catalysis involves fermenting glucose into L-alanine and then converting the L-alanine into DL-alanine by alanine racemase. The method requires a long production cycle and is rather expensive. Therefore, its application in mass production is challenging. With glucose as a substrate, DL-alanine can be directly produced by fermentation with a strain constructed by genetic engineering. This method has attracted widespread attention thanks to the advantages of inexpensive and easily available raw materials, high product yield, low cost and so on.

Compared with fermentation at a common temperature of 42° C. or lower, high-temperature fermentation can provide a significantly reduced risk of contamination, improved raw material conversion efficiency and reduced heat exchange cost and is therefore of high value to large-scale fermentation applications. In production of alanine by fermentation, genetically engineered Escherichia coli (Escherichia coli) strains are typically used as producing strains. However, as the optimum growth temperature for these strains is 37° C., they are not suitable for use in high-temperature fermentation.

Therefore, those skilled in the art are directing their effort toward developing a strain suitable for large-scale production of DL-alanine by fermentation under a high-temperature condition.

### SUMMARY OF THE INVENTION

In view of the above, the problem sought to be solved by the present invention is to provide a genetically engineered strain capable of producing DL-alanine under a high-temperature condition of 42° C. or higher, as well as a method of construction and use thereof.

To achieve the above goal, in a first aspect of the present invention, there is provided a method of constructing a DL-alanine-producing genetically engineered strain, which includes steps of:
providing an original strain possessing a pyruvate synthesis pathway;
constructing a genetically engineered strain for a two-step method by engineering a genome of the original strain through steps S200 and S300, or
constructing a genetically engineered strain overexpressing an alanine racemase gene through steps S200, S300 and S400;
S200: inserting a copy of a 6-phosphofructokinase gene pfk and a copy of a pyruvate kinase gene pyk;
S300: inserting a gene GSald for alanine dehydrogenase thermostable at 42° C. to 55° C.;
S400: inactivating or deleting an alanine racemase gene and introducing an overexpressed alanine racemase gene.

A DL-alanine-producing genetically engineered strain constructed in accordance with the first aspect of the present invention is capable of DL-alanine production by fermentation under a high-temperature condition of 42° C. to 55° C.

In some embodiments of the present invention, the original strain further possesses a lactate synthesis pathway and the genome of the original strain contains a lactate dehydrogenase gene. In these cases, the method further includes the step of:
S100: inactivating or deleting the lactate dehydrogenase gene in the genome of the original strain.

In some embodiments of the present invention, in step S400, an alanine racemase gene alr1 and an alanine racemase gene alr2 are completely inactivated or deleted.

Additionally or alternatively, in step S400, the introduction of the overexpressed alanine racemase gene is accomplished by inserting a strong promoter and alanine racemase gene(s), and the inserted alanine racemase gene(s) is/are an alanine racemase gene alr1 or/and an alanine racemase gene alr2.

In a second aspect of the present invention, there is provided a genetically engineered strain constructed in accordance with the method of the first aspect of the present invention.

In a third aspect of the present invention, there is provided use of the genetically engineered strain according to the second aspect of the present invention for DL-alanine production.

In a fourth aspect of the present invention, there is provided a method of producing DL-alanine, in which the genetically engineered strain according to the second aspect of the present invention is subjected to fermentation culture, wherein the fermentation culture is at a temperature of 42° C. to 55° C.

In some embodiments of the present invention, the fermentation culture is conducted in a manner selected from any one of:
using a genetically engineered strain for a two-step method, wherein the method comprises a fermentation culture step and an alanine racemase treatment step; or
using a genetically engineered strain with an overexpressed alanine racemase gene, wherein the method comprises a fermentation culture step and does not include an additional alanine racemase treatment step; or
using a genetically engineered strain with the overexpressed alanine racemase gene, wherein the method comprises a fermentation culture step and further comprises an alanine racemase treatment step.

Benefits offered by the techniques disclosed in the present invention include:

With a thermophilic strain as an original strain, a strain can be constructed, which is capable of producing DL-alanine at a high temperature of 42° C. to 55° C. (e.g., 50° C.). Through enhancing the glycolysis pathway or/and introducing thermostable alanine dehydrogenase, a genetically engineered strain for use in a two-step method can be constructed. Through adding microbial alanine racemase after fermentation with this strain, high-yield production of racemic DL-alanine can be achieved. Another strain capable of producing racemic DL-alanine using a direct fermentation method can be constructed by inactivating or deleting alanine racemase genes in the genetically engineered strain for use in the two-step method and then introducing overexpressed alanine racemase gene(s). This entails a simplified process with lower cost. Coupled with addition of exogenous alanine racemase, more precise optical purity control of racemic DL-alanine is enabled. In case of the original strain possessing a lactate synthesis pathway, through blocking this lactate synthesis pathway, the proportion of a pyruvate synthesis pathway can be augment, resulting in a higher yield of DL-alanine. Combining the above strategies can synergistically achieve high-yield production of racemic DL-alanine under a high-temperature condition.

Genetically engineered strains constructed in accordance with, and fermentation processes provided in, the present invention overcome the problems of fermentation at a low temperature, high cost and the like, which arise from the use of conventional DL-alanine production techniques, and enable high-temperature (e.g., 42° C. to 55° C.) production of DL-alanine. The fermentation processes provide the advantages including low energy consumption and resistance to contamination, can effectively lower down production cost and are therefore of high value in industrial application. Further, they enable high-yield production of racemic DL-alanine and are promising in future applications. A DL-alanine yield of 120 g/L or higher, or even 130 g/L or higher, can be obtained. The produced DL-alanine is nearly racemic, and the optical purity of L-alanine therein is very close to 50%. In particular, the optical purity of L-alanine can be substantially controlled within the range of 49.5% to 50.5%.

For example, Bacillus licheniformis usable in fermentation at a high temperature of 50° C. can be used for this purpose. With a Bacillus licheniformis BN11 strain capable of high-yield production of D-lactate as a starting strain, a D-lactate synthetic pathway therein may be eliminated to enhance the flux of the glycolysis pathway. Moreover, thermostable heterologous alanine dehydrogenase may be introduced, and an endogenous D-alanine synthesis pathway may be eliminated. Additionally, overexpressed alanine racemase gene(s) may be introduced. In this way, a genetically engineered strain can be constructed, which is capable of producing racemic DL-alanine by direct fermentation under a high-temperature condition.

Below, the concept, structural details and resulting technical effects of the present application will be further described with reference to the accompanying drawings to provide a full understanding of the objects, features and effects of the invention.

### BRIEF DESCRIPTION OF THE DRAWING

Other features, objects and advantages of the present invention will become more apparent upon reading the following detailed description of non-limiting examples when taken in conjunction with the accompanying drawings, in which:

FIG. 1 schematically illustrates alanine synthesis and metabolism pathways in an exemplary genetically engineered Bacillus licheniformis strain according to an embodiment of the present invention;

FIG. 2 is a plot diagram of DL-alanine production by fed-batch fermentation at 50° C. in a 5-L fermenter using a DL-alanine-producing genetically engineered strain BDLA-1 according to an embodiment of the present invention (▴: OD600; ▪: glucose; ●: alanine);

FIG. 3 is a plot diagram of fermentation of DL-alanine at 50° C. in a 5-L fermenter by a DL-alanine-producing genetically engineered strain BDLA-1 using a two-step method according to an embodiment of the present invention (▴: OD600; ▪: glucose; ●: alanine); and

FIG. 4 shows the structure of a pKVM vector used in some embodiments of the present invention, which contains two antibiotic resistance genes: ampR (ampicillin resistance) and ermC (erythromycin resistance), as well as the bgaB gene that encodes galactosidase. All of these genes can be used as selectable markers. This plasmid has a heat-sensitive origin of replication (oriT pE194ts) and origins of replication in Escherichia coli (ori) and Bacillus licheniformis (oriT).

### DETAILED DESCRIPTION OF THE PREFERRED EMBODIMENTS

Specific embodiments of the present invention will be described in detail below with reference to the accompanying drawings.

In the following, the figures annexed to this specification are referenced to describe a few preferred embodiments and examples of the present invention so that the techniques thereof become more apparent and readily understood. The invention may be embodied and exemplified in many different forms, and its scope is not limited to the embodiments and examples herein.

It will be understood that the features described hereinabove and the features detailed in the following description (including, but not limited to, the embodiments and examples) can be combined in any suitable manner to create new embodiments, as long as the invention can be implemented.

As used herein, the terms “preferred”, “particular”, “specific”, “further”, “furthermore” and the like are used merely to describe embodiments or examples with good effects or with certain degrees of particularity. It will be understood that they are not intended to limit the scope of the invention in any way.

As used herein, “and/or” and “or/and” both means that the listed items are alternatives, but the alternatives also include any combination of the listed items.

As used herein, “a combination thereof”, “any combination”, “an arbitrary combination” and “in any combination” all mean that the listed items can be combined in any suitable manner as long as the present invention can be implemented. The number of items that can be combined is two or more.

As used herein, the term “plurality” means “two or more”.

As used herein, any reference to a percentage concentration should be understood to refer to a final concentration in a system, unless otherwise defined or specified.

As used herein, any numerical range recited is intended to include both the lower and upper limits, unless otherwise specified.

As used herein, “or higher” or “or lower” following a number is intended to refer to a range including the specific number, unless otherwise defined.

The features of the present invention described above and below can be combined in any suitable manner, as long as there is no conflict and the resultant embodiment can be implemented to solve the problem that the present invention seeks to solve. The features can be combined in any suitable manner, as long as the resultant embodiment can solve the problem that the present invention seeks to solve and achieve an expected effect.

As used herein, “starting strain” and “original strain” have the same meaning and can be used interchangeably.

As used herein, any reference to temperature control should be understood to mean that a temperature is controlled so as to remain at a constant value or vary within a range.

As used herein, the term “high-temperature”, when used to describe the production of DL-alanine, is relative to a common temperature (lower than 42° C., typically 37° C. or lower). As used herein, unless otherwise defined, “high-temperature production” is intended to refer to production by fermentation at 42° C. to 55° C., for example, but not limited to, 42° C., 43° C., 44° C., 45° C., 46° C., 47° C., 48° C., 49° C., 50° C., 52° C., 53° C., 54° C. or 55° C.

As used herein, unless otherwise specified, “pyruvate synthesis pathway” refers to a pathway that synthesizes pyruvate by glycolysis of a carbon source. As used herein, “possessing a pyruvate synthesis pathway” means “having the ability to synthesize pyruvate endogenously”.

As used herein, unless otherwise specified, “lactate synthesis pathway” refers to a synthetic pathway that converts pyruvate to lactate. As used herein, unless otherwise specified, “possessing a lactate synthesis pathway” means “having the ability to producing lactate through a lactate synthesis pathway”. That is, “possessing a lactate synthesis pathway” means “having the ability to synthesize lactate endogenously”.

As used herein, unless otherwise specified, “alanine synthesis pathway” refers to a synthetic pathway that converts pyruvate to alanine. As used herein, unless otherwise specified, “possessing an alanine synthesis pathway” means “having the ability to producing alanine through an alanine synthesis pathway”, i.e., “having the ability to synthesize alanine endogenously”. As used herein, when something is described as possessing an alanine synthesis pathway, unless otherwise specified, it is intended to mean that it possesses both a pathway that synthesizes L-alanine from pyruvate and a pathway that synthesizes D-alanine from L-alanine.

As used herein, unless otherwise specified, “D-alanine synthesis pathway” refers to a pathway that synthesizes D-alanine from L-alanine, and “possessing a D-alanine synthetic pathway” means “having the ability to synthesize D-alanine endogenously”.

As used herein, a gene cluster fragment refers to a fragment containing at least two genes, which may be either identical or different. When describing a gene cluster fragment composed of identical genes, the term “repeated” shall be broadly interpreted as meaning that the genes may be directly adjacent to one another, or a spacer sequence may be present between any two of the genes.

According to the present invention, one of the typical approaches for deleting a gene is to knock it out.

TABLE 1
Abbreviations and Full Names in
English and Chinese of Some Terms

| Abbreviation | English | Chinese |
|---|---|---|
| NADH | Nicotinamide Adenine | [symbol] [symbol] [symbol] |
| | Dinucleotide Hydrogen | |

TABLE 2
Names of Some Terms in English and Chinese

| English | Chinese |
|---|---|
| Geobacillus stearothermophilus | [symbol] [symbol] |
| Bacillus licheniformis | [symbol] |
| Bacillus coagulans | [symbol] |
| Glucose | [symbol] |
| Alanine | [symbol] |

Amino acid and nucleotide sequences mentioned herein are summarized in Table 3.

TABLE 3

| Sequence | Type of | Species of | Characteristic |
|---|---|---|---|
| No. | Sequence | Organism | Information |
| SEQ ID | DNA | Artificial | Alanine Dehydrogenase |
| NO. 1 | | Sequence | Gene (GSald) |
| SEQ ID | DNA | Bacillus | Alanine Racemase |
| NO. 2 | | licheniformis | Gene (alr1) |
| SEQ ID | DNA | Bacillus | Alanine Racemase |
| NO. 3 | | licheniformis | Gene (alr2) |
| SEQ ID | DNA | Artificial | Pₐₗₛ Promoter |
| NO. 4 | | Sequence | |
| SEQ ID | DNA | Artificial | pKVM-XPDdldh-UpF Primer |
| NO. 5 | | Sequence | |
| SEQ ID | DNA | Artificial | XPDdldh-UpR Primer |
| NO. 6 | | Sequence | |
| SEQ ID | DNA | Artificial | XPDdldh-DownF Primer |
| NO. 7 | | Sequence | |
| SEQ ID | DNA | Artificial | pKVM-XPDdldh-DownR Primer |
| NO. 8 | | Sequence | |
| SEQ ID | DNA | Artificial | pKVM-XPDldh-UpF Primer |
| NO. 9 | | Sequence | |
| SEQ ID | DNA | Artificial | Pₐₗₛ-XPDldh-UpR Primer |
| NO. 10 | | Sequence | |
| SEQ ID | DNA | Artificial | XPDldh-Pₐₗₛ-F Primer |
| NO. 11 | | Sequence | |
| SEQ ID | DNA | Artificial | PFYAK-Pₐₗₛ-R Primer |
| NO. 12 | | Sequence | |
| SEQ ID | DNA | Artificial | Pₐₗₛ-PFYAK-F Primer |
| NO. 13 | | Sequence | |
| SEQ ID | DNA | Artificial | XPDldh-PFYAK-R Primer |
| NO. 14 | | Sequence | |
| SEQ ID | DNA | Artificial | PFYAK-XPDldh-DownF Primer |
| NO. 15 | | Sequence | |
| SEQ ID | DNA | Artificial | pKVM-XPDldh-DownR Primer |
| NO. 16 | | Sequence | |
| SEQ ID | DNA | Artificial | Pₐₗₛ-XPDdldh-UpR Primer |
| NO. 17 | | Sequence | |
| SEQ ID | DNA | Artificial | XPDdldh-Pₐₗₛ-F Primer |
| NO. 18 | | Sequence | |
| SEQ ID | DNA | Artificial | XPDdldh-GSald-R Primer |
| NO. 19 | | Sequence | |
| SEQ ID | DNA | Artificial | GSald-XPDdldh-DownF Primer |
| NO. 20 | | Sequence | |
| SEQ ID | DNA | Artificial | pKVM-XPDalr1-UpF Primer |
| NO. 21 | | Sequence | |
| SEQ ID | DNA | Artificial | XPDalr1-UpR Primer |
| NO. 22 | | Sequence | |
| SEQ ID | DNA | Artificial | XPDalr1-DownF Primer |
| NO. 23 | | Sequence | |
| SEQ ID | DNA | Artificial | pKVM-XPDalr1-DownR Primer |
| NO. 24 | | Sequence | |
| SEQ ID | DNA | Artificial | pKVM-XPDalr2-UpF Primer |
| NO. 25 | | Sequence | |
| SEQ ID | DNA | Artificial | XPDalr2-UpR Primer |
| NO. 26 | | Sequence | |
| SEQ ID | DNA | Artificial | XPDalr2-DownF Primer |
| NO. 27 | | Sequence | |
| SEQ ID | DNA | Artificial | pKVM-XPDalr2-DownR Primer |
| NO. 28 | | Sequence | |
| SEQ ID | DNA | Artificial | pKVM-XPDPdh-UpF |
| NO. 29 | | Sequence | |
| SEQ ID | DNA | Artificial | Pals-XPDPdh-UpR |
| NO. 30 | | Sequence | |
| SEQ ID | DNA | Artificial | XPDPdhUp-Pals-F |
| NO. 31 | | Sequence | |
| SEQ ID | DNA | Artificial | Alr1-Pals-R |
| NO. 32 | | Sequence | |
| SEQ ID | DNA | Artificial | Pals-alr1-F |
| NO. 33 | | Sequence | |
| SEQ ID | DNA | Artificial | XPDPdhDown-alr1-R |
| NO. 34 | | Sequence | |
| SEQ ID | DNA | Artificial | Alr1-XPDPdh-DownF |
| NO. 35 | | Sequence | |
| SEQ ID | DNA | Artificial | pKVM-XPDPdh-DownR |
| NO. 36 | | Sequence | |
| SEQ ID | DNA | Artificial | Alr2-Pals-R |
| NO. 37 | | Sequence | |
| SEQ ID | DNA | Artificial | Pals-alr2-F |
| NO. 38 | | Sequence | |
| SEQ ID | DNA | Artificial | XPDPdhDown-alr2-R |
| NO. 39 | | Sequence | |
| SEQ ID | DNA | Artificial | Alr2-XPDPdh-DownF |
| NO. 40 | | Sequence | |
| SEQ ID | DNA | Bacillus | 6-Phosphofructokinase |
| NO. 41 | | coagulans | Gene (pfk) |
| SEQ ID | DNA | Bacillus | Pyruvate Kinase Gene pyk |
| NO. 42 | | coagulans | |
| SEQ ID | DNA | Bacillus | D-Lactate Dehydrogenase |
| NO. 43 | | licheniformis | Gene (ldh_{Ti}) |
| SEQ ID | PRT | Artificial | Alanine Dehydrogenase |
| NO. 44 | | Sequence | Gene (GSald) |

SEQ ID NO. 1 (GSald):
ATGAAAATCGGCATCCCGAAAGAAATCAAAAACAACGAAAACCGCGTCGC
GATCACGCCGGCGGGCGTCATGACGCTGGTCAAAGCGGGCCATGACGTCT
ATGTCGAAACGGAAGCGGGCGCGGGCAGCGGCTTTAGCGACAGCGAATAT
GAAAAAGCGGGCGCGGTCATCGTCCCGAACGCGGAAGACGCGTGGACGGC
GGAAATGGTCCTGAAAGTCAAAGAACCGCTGGCGGAAGAATTTCGCTATT
TTCGCCCGGGCCTGATCCTGTTTACGTATCTGCATCTGGCGGCGGCGGAA
GCGCTGACGAAAGCGCTGGTCGAACAGAAAGTCGTCGGCATCGCGTATGA
AACGGTCCAGCTGGCGAACGGCAGCCTGCCGCTGCTGACGCCGATGAGCG
AAGTCGCGGGCCGCATGAGCGTCCAGGTCGGCGCGCAGTTTCTGGAAAAA
CCGCATGGCGGCAAAGGCATCCTGCTGGGCGGCGTCCCGGGCGTCCGCCG
CGGCAAAGTCACGATCATCGGCGGCGGCACGGCGGGCACGAACGCGGCGA
AAATCGCGGTCGGCCTGGGCAGCGACGTCACGATCCTGGACATCAACGCG
GAACGCCTGCGCGAACTGGACGACCTGTTTGGCGACCATGTCACGACGCT
GATGAGCAACAGCTATCATATCGCGGAATGCGTCCGCGAAAGCGACCTGG
TCGTCGGCGCGGTCCTGATCCCGGGCGCGAAAGCGCCGAAACTGGTCACG
GAAGAAATGGTCCGCAGCATGACGCCGGGCAGCGTCCTGGTCGACATCGC
GATCGACCAGGGCGGCATCTTTGAAACGACGGACCGCGTCACGACGCATG
ACGACCCGACGTATGTCAAACATGGCGTCGTCCATTATGCGGTCGCGAAC
ATGCCGGGCGCGGTCCCGCGCACGAGCACGTTTGCGCTGACGAACGTCAC
GATCCCGTATGCGCTGCAGATCGCGAACAAAGGCTATCGCGCGGCGTGCC
TGGACAACCCGGCGCTGCTGAAAGGCATCAACACGCTGGACGGCCATATC
GTCTATGAAGCGGTCGCGGCGGCGCATAACATGCCGTATACGGACGTCCA
TAGCCTGCTGCATGGCTAA
SEQ ID NO. 2 (Alr1):
ATGATGAGCTTAAAACCATTCTATAGAAAGACATGGGCCGAAATCGATTT
AACGGCTTTAAAAGAAAACGTCCGCAATATGAAGCGGCACATCGGCGAGC
ATGTCCGCCTGATGGCCGTCGTTAAAGCGAATGCCTACGGACACGGGGAT
GCACAGGTAGCGAAGGCGGCTCTTGCAGAAGGGGCGTCCATTCTTGCTGT
GGCTTTATTGGATGAAGCGCTTTCGCTGAGGGCGCAGGGGATTGAAGAAC
CGATTCTTGTCCTCGGTGCAGTGCCGACCGAATATGCAAGCATTGCCGCG
GAAAAGCGCATTATCGTGACTGGCTACTCCGTCGGCTGGCTGAAAGACGT
GCTCGGTTTTCTGAATGAGGCCGAAGCTCCTCTTGAATATCATTTGAAGA
TCGACACGGGCATGGGCCGCCTTGGCTGCAAAACGGAAGAAGAGATCAAA
GAAATGATGGAGATGACCGAATCGAACGATAAGCTCAATTGTACGGGCGT
GTTCACTCATTTCGCCACGGCGGACGAAAAGGACACCGATTATTTCAACA
TGCAGCTTGACCGCTTTAAAGAGCTGATCAGCCCCCTCCCGCTTGACCGT
TTGATGGTGCATTCGTCAAACAGCGCCGCGGGTCTGCGCTTCAGGGAACA
GCTATTTAATGCCGTCCGCTTCGGCATCGGCATGTACGGTTTGGCGCCGT
CAACCGAAATAAAAGACGAGCTGCCGTTTCGTCTGCGGGAAGTGTTTTCG
CTTCATACCGAACTCACCCATGTGAAAAAAATTAAAAAAGGCGAGAGCGT
CAGCTACGGGGCGACATATACAGCTCAGCGCGACGAATGGATCGGGACAG
TCCCCGTCGGGTATGCCGACGGATGGCTGAGGCGCCTGGCCGGAACGGAA
GTGCTGATCGACGGAAAACGCCAAAAAATAGCAGGGAGAATCTGCATGGA
CCAGTTCATGATTTCCCTTGCCGAAGAATACCCTGTCGGCACAAAGGTTA
CCTTGATCGGAAAGCAAAAAGACGAATGGATCTCAGTCGACGAAATCGCC
CAAAATTTGCAGACGATCAATTATGAAATTACCTGTATGATAAGTTCAAG
GGTGCCCCGTATGTTTTTGGAAAATGGGAGTATAATGGAAATAAGGAATC
CGATCTTGCCTGATCAATCCTGA
SEQ ID NO. 3 (Alr2):
ATGAAAAAGCTTTGCCGTGAAGTTTGGGTAGAGGTAAATCTTGATGCGAT
CAAAAAAAATTTGCGCGCGTTTCGGCGGCATATTCCGAAAAAGAGCAAAA
TTATGGCTGTCGTAAAAGCGAATGCTTATGGTCACGGATCGGTGGAAGTT
GCACGCCATGCACTTGAACATGGTGCGAGTGAGCTCGCCGTTGCCTCGGT
GGAGGAAGCGGTCGTTTTACGAAAAGCGGGGATTAAAGCGCCGATCCTTG
TGCTTGGTTTCACCCCGCTGAGCTGTGTGAAAGAAGCGGCAGCTTGGAAT
ATATCGTTATCAGCTTTTCAAGTTGACTGGATTAAAGAAGCGAACGAGAT
ATTGGAAAATGAAGCAGATCCTAACCGGCTGGCTGTTCATATCAATGTGG
ATACCGGCATGGGGCGTTTAGGTGTACGAACAAAGGAAAAGCTTTTAGCA
ATCGTGGAAGCGCTGACGGCAAGTGAAAACCTCGAATGGGAAGGAATTTT
TACGCATTTTTCCACAGCTGACGAACCGGATACTGAGCTAACCATGATTC
AACACGAAAAGTTTATCAGCTTTCTTCGCTTTCTGAAAGAACAAGGCTTT
AAGCTGCCTACGGTGCATATGAACAATACGGCCGCGGCGATCGCTTTTCC
GGAATTCAGCGCTGATATGATTCGCTTAGGCATCGGAATGTATGGATTAT
ATCCTTCCGATTATATCAGGCAGCTTAATCTCGTTAAGCTTGTGCCTGCA
CTAAGCTTGAAGGCGCGAATCGCTTATGTGAAAACCATGTTGACTGAACC
GCGGACGGTTAGTTATGGTGCTACATATGTTGCAGAGCGCGGGGAAGTCA
TTGCCACAATTCCGGTCGGCTATGCTGACGGCTATTCCCGTGAACTTTCC
AACCGCGGTTTTATTCTTCATCGAGGAAGACGAGTGCCGGTGGCGGGAAG
AGTAACAATGGATATGATAATGGTCAGTCTGGGAGAGGGTGAAGGTAAAC
AAGGAGAGGAAGTCGTGATTTACGGCCGGCAAAAGGGAGCAGAGATATCT
GTTGATGAAATTGCGGAAATGCTTGATACGATCAACTATGAAGTGGTATC
TACCATAAGCTGGCGCGTCCCTCGTTTTTATATAAGAGACGGCGAGATTT
TTAAAAAGTCGACCCCGCTGTTATACGTGTAG
SEQ ID NO. 4 (Pₐₗₛ promoter):
AAGGTGACGCCTATTTCACTTTCTAGCTGTTTAATCTGCTGGCTGAGCGG
AGGCTGAGTCATGTTCAGCCGAAGAGCTGCTTTTCCGAAATGCAGTTCTT
CGGCAACAACCATAAAATAACGAAGATGGCGCAGCTCCATTAATCACTCA
TTCCTTTCTGAATGCGATTTCAGTCGTTTTACATATTAATTGTAAGACAA
AGAAGTATTGGAAAACAATTTCCACAAGATGTATATTTAATAATACAATA
ATTTTATTAAAAATTCATTGTAAATGAATGAAAATGGAGGAGTGAGGGCT
SEQ ID NO. 5:
CCTCGCGTCGGGCGATATCGGATCCGAAGGGGAAAGTCTTCGATTTCT
SEQ ID NO. 6:
AGAGGGCTTTTTCATGCTGAAGAGGTCAAAAAGAGCC
SEQ ID NO. 7:
TTTGACCTCTTCAGCATGAAAAAGCCCTCTTTGAAAAG
SEQ ID NO. 8:
CCATGGTACCCGGGAGCTCGAATTCCATAAGACCGCTGATGACAAGC
SEQ ID NO. 9:
TCCAGCCTCGCGTCGGGCGATATCGTCCCCATAACAACGGAATCATC
SEQ ID NO. 10:
AATAGGCGTCACCTTGACTCATCATTCCTTTGCCGTT
SEQ ID NO. 11:
AAGGAATGATGAGTCAAGGTGACGCCTATTTCACTTTC
SEQ ID NO. 12:
TCCAATTCGCTTCATAGCCCTCACTCCTCCATTTTC
SEQ ID NO. 13:
GGAGGAGTGAGGGCTATGAAGCGAATTGGAGTATTGACA
SEQ ID NO. 14:
CTTCATGGTGTTCAGTTACAATACAGTCGCATGGCC
SEQ ID NO. 15:
GCGACTGTATTGTAACTGAACACCATGAAGATACTAACATCA
SEQ ID NO 16:
ACTAGACAGATCTATCGATGCATGCTTTCCCTTATTCCTTTAAACCCG
SEQ ID NO. 17:
AATAGGCGTCACCTTGCTGAAGAGGTCAAAAAGAGCC
SEQ ID NO. 18:
TTTGACCTCTTCAGCAAGGTGACGCCTATTTCACTTTCT
SEQ ID NO. 19:
AGAGGGCTTTTTCATTTAGCCATGCAGCAGGCTATG
SEQ ID NO. 20:
CTGCTGCATGGCTAAATGAAAAAGCCCTCTTTGAAAAG
SEQ ID NO. 21:
CCTCGCGTCGGGCGATATCGGATCCAAAATATGACGCTGTCTCAAATTGA
SEQ ID NO. 22:
CTAATTCATCAATTTGACACTTCCTGTTCCTTGTTTCACT
SEQ ID NO. 23:
GGAACAGGAAGTGTCAAATTGATGAATTAGCGGAAAAAC
SEQ ID NO. 24:
CCATGGTACCCGGGAGCTCGAATTCCGGAGTCTCTTTCAAAACCGTAG
SEQ ID NO. 25:
CCTCGCGTCGGGCGATATCGGATCCAAAATCATGTAAGCCCATTCCG
SEQ ID NO. 26:
GTGAGTATGGGAAAACAACGCTCCCTTCTTTCTTGTC
SEQ ID NO. 27:
AAGAAGGGAGCGTTGTTTTCCCATACTCACAGGCCG
SEQ ID NO. 28:
CCATGGTACCCGGGAGCTCGAATTCTAAAATGAAGGTGGTCCGGGAT
SEQ ID NO. 29:
CCTCGCGTCGGGCGATATCGGATCCAAGTGAAAGGCGAAGAGCAAAC
SEQ ID NO. 30:
AATAGGCGTCACCTTTTATTTTACAACGTGGATCGGC
SEQ ID NO. 31:
CACGTTGTAAAATAAAAGGTGACGCCTATTTCACTTTCT
SEQ ID NO. 32:
TTTTAAGCTCATCATAGCCCTCACTCCTCCATTTTC
SEQ ID NO. 33:
GGAGGAGTGAGGGCTATGATGAGCTTAAAACCATTCTATAGAA
SEQ ID NO. 34:
GAGGGGGTTTTTGATTCAGGATTGATCAGGCAAGATC
SEQ ID NO. 35:
CCTGATCAATCCTGAATCAAAAACCCCCTCTGCAG
SEQ ID NO. 36:
CCATGGTACCCGGGAGCTCGAATTCATATTATTGTGCCGAAC
SEQ ID NO. 37:
GCAAAGCTTTTTCATAGCCCTCACTCCTCCATTTTC
SEQ ID NO. 38:
GGAGGAGTGAGGGCTATGAAAAAGCTTTGCCGTGAA
SEQ ID NO. 39:
GAGGGGGTTTTTGATCTACACGTATAACAGCGGGGTC
SEQ ID NO. 40:
CTGTTATACGTGTAGATCAAAAACCCCCTCTGCAG
SEQ ID NO. 41 (Pfk):
ATGAAGCGAATTGGAGTATTGACAAGCGGCGGCGATGCACCGGGGATGAA
TGCGGCGGTCCGCGCGATTGCCCGTAAAGGGATTTATCACGGCCTGGAAG
TTTACGGCATTCGCCAAGGTTATAACGGATTGATTCAAGGAAACATCCAA
AAGCTCGAAGCAGGATCTGTTGGCGATATTCTCCAGCGGGGCGGCACGGT
TTTGCAGTCGGCAAGAAGCGAAGAATTCAAAACGCCGGAAGGGCAGCAAA
AAGCGATCAGGCAGCTGAAAGACCATGGCATTGAAGCGCTCGTTGTGATC
GGCGGCGACGGTTCCTACCAAGGGGCCAAAAAGTTGACGGAACAGGGCTT
TAACTGCATTGGTGTGCCAGGGACAATCGATAACGACATCCCGGGGACGG
ATTTTACAATCGGTTTTGATACGGCATTGAACACAGTGCTTGATGCGATT
GATAAAATTCGCGACACCGCTTCTTCCCACGAACGCACCTTTATTATTGA
AGTCATGGGCAGAAATGCCGGGGATATCGCGCTCTGGTCCGGCCTGGCCG
GCGGAGCCGAATCGATTATTATTCCGGAAGAAAAATATGACTTAAAAGAT
GTCGTGGAGCGTCTTGAACAGGGGAGAAAACGCGGCAAACGCCACAGCAT
CATCATTGTCGCGGAAGGCGTGATGAGCGGCAACGAGTTTGCTGAACAAT
TGAAAAAAACCGGTGTGATCGGCGATACCCGCGTTTCTGTTCTCGGCCAT
ATCCAGCGCGGCGGTTCTCCGACGGCATTTGACCGCGTGCTTGCAAGCCG
CCTCGGCGCAAGGGCTGTTGAACTGCTGCTTGAAGGAAAAGGGGGCCGCG
CTGTCGGCATTCAAAATAACCAGCTGGTTGACCACGATATCCTTGAGATT
CTCGGAAAACCGCACGCCGTTAATAAAAACATGTACAAGCTGTCGAAAGA
ATTGTCGATCTAA
SEQ ID NO. 42 (Pyk):
ATGAAAAAAACCAAAATTGTATGTACAATCGGACCTGCCAGTGAAAGTGT
GGAAATGCTTGAAAGATTAATGGCAAACGGGATGGATGTTTGCCGCCTGA
ACTTCTCGCACGGCAGCCATGAGGAACATCTTGCCCGGATTAAAAATATC
CGTGAAGCTGCAAAAAACCAAAACAAAACGATCGGGCTTCTGCTCGATAC
AAAGGGCCCGGAAATCCGCACCCATGATATGAAAGACGGCGGATTCGAGC
TCGTTGAAGGCATGACACCGGTCATTTCAATGACAGAAGTGCTCGGGACA
CCGGAAAAATTTTCGGTCACATATGAAGGGCTGATTGATGATGTGCACGT
TGGCTCTAAAATTTTACTTGATGACGGTTTGATTGAACTGGAAGTGACGG
CCATCGATAAAAACGCCGGTGAAATCCATACAAAAGTGCTGAACCGCGGC
GTTTTGAAAAACAAAAAAGGTGTTAACGTCCCGGGTGTTTCCGTGAACCT
TCCGGGCATCACCGAAAAAGACGTGAGCGATATCCTGTTCGGGCTTGAAC
AAGGCATTGACTTCATTGCGGCTTCGTTTGTACGCCGGCCGTCCGACGTT
TTGGAAATCCGCCAGCTCCTTGAAGAACACGATGCTTTGCATGTGAAAAT
TTTCCCTAAAATTGAAAACCAGGAAGGCGTCGACAATATCGATGAAATCC
TTGCGGTATCAGACGGCTTAATGGTTGCCCGCGGCGACCTCGGCGTTGAA
ATTCCGACCGAAGCGGTGCCGCTCGTACAAAAAGAAATGATCAGAAAATG
TAATACGCTCGGCAAACCGGTGATTACCGCAACGCAAATGCTTGATTCGA
TGCAACGCAACCCGCGCCCGACCCGCGCGGAAGCAAGCGACGTGGCCAAC
GCCATTTTTGACGGCACGGATGCGATCATGCTTTCCGGCGAAACGGCAGC
CGGGAAATATCCTGCTGAAGCGGTTAAGACGATGTACAATATTGCGGTTC
ATGTGGAAAAAGCAATTAACCATCGCGATATTCTGAACAAGCGCAGCAAG
AGCACGGACCATAATATGACAGACGCTATCTGCCAGTCCGTTGCCCATAC
GGCTTTAAATCTTGATGTGAATGCCATTATTGCGCCGACTGAAAGCGGCT
ATACGGCACGCATGATCTCCAAATACCGCCCGGCGGCCCCAATCATTGCT
GTCACGAGCGATCCGAAAGTACAACGCGGCTTAACTGTTGTGTCCGGCGT
ATACCCACAATTGGGCACAAAGGCAAACAATACGGATGAAATGCTTGAAA
TTGCAGTGGAGGAAGCGTTGAAATCCGAAATCGTCCATCACGGCGACCTT
GTGATCATTACAGCAGGCGTCCCGGTTGGTGGGAAAGGCACCACCAACCT
GATGAAAGTGCACCTGATCGGTGATATATTGGCAAAAGGCCAGGGAATCG
GCAGAAAATCGGCATTCGGCCCGGTCATCGTTGCTGAAAGCCCTGAAGAA
GCAAACGCAAAGGCAACAGAAGGTTGTGTGCTCGTCACGAGAACGACCGA
CAAAGAAATCATGCCGGCCATTGAAAAATGCGCCGCGCTGATTACGGAAG
AAGGCGGCTTGACAAGCCATGCTGCAGTTGTTGGCATCAATGTCGGCATT
CCGGTCATTGTTGGCGTTGAAAAAGCCGTTTCCATTTTTGAAGACGGGCA
GGAAGTTACGGTAGATGCGGCAACCGGCTCGGTTTACAACGGCCATGCGA
CTGTATTGTAA
SEQ ID NO. 43 (Ldhti):
ATGAAAGTAATTTTTTTTTCTATGCACCCGTATGAAGAGGAATTTCTGGG
TCCGATTCTGCCGTCTGACTGGGACGTAGAAATGACCCCGGACTTTCTGG
ACGAAACCACCGTGGAAAAGGCTAAAGGTGCCCAGGTAGTAAGCCTGTTT
GTTTCTGACAAAGCTGATGGTCCGGTACTGGAAGCGCTGCATTCTTACGG
TGTGGGCCTGCTGGCCCTGCGTTCTGCTGGCTATGATCACATCGATATTG
AGACCGCAAAACGCCTGGGTATCAAAGTAGTTAACGTGCCAGCCTATTCT
CCGCACGCTATCGCTGACCATACTCTGGCTATCATGCTGGCTCTGATTCG
TCGTCTGCACCGTGCCCATGATAAAGTGCGCCTGGGTGATTTTGATCTGG
ATGGTCTGATGGGCTTTGATCTGAACGGCAAAGTTGCTGGTGTAATTGGT
CTGGGTAAAATCGGTCGCCTGGTAGCTACCCGCCTGAAAGCGTTTGGTTG
CAAAGTTCTGGGCTATGATCCATACATTCAGCCGGAAATCGTAGAAAACG
TTGATCTGGATACCCTGATCACTCAGGCTGATATCATTTCTATTCATTGT
CCGCTGACCCGTGAAAACTTTCATATGTTTAACGAAGAGACTTTTAAGCG
TATGAAACCGGGTGCTATTCTGGTTAACACCGCGCGTGGTGGTCTGATCG
ATACCAAGGCCCTGCTGGAGGCCCTGAAGTCTGGTAAACTGGGCGGCGCA
GCCCTGGATGTGTATGAATATGAACGTGGCCTGTTTTTTAAAAACCACCA
AAAAGAAGGTATCAAAGACCCGTATCTGGCCCAGCTGCTGGGTCTGGCCA
ACGTAGTGCTGACCGGTCATCAGGCCTTTCTGACCCGTGAGGCTGTAAAA
AACATCGAAGAAACTACCGTAGAAAACATTCTGGAATGGCAAAAGAACCC
GCAGGCAAAGCTGAAAAACGAAATCTAA
SEQ ID NO. 44 (GSald):
MKIGIPKEIKNNENRVAITPAGVMTLVKAGHDVYVETEAGAGSGFSDSEY
EKAGAVIVPNAEDAWTAEMVLKVKEPLAEEFRYFRPGLILFTYLHLAAAE
ALTKALVEQKVVGIAYETVQLANGSLPLLTPMSEVAGRMSVQVGAQFLEK
PHGGKGILLGGVPGVRRGKVTIIGGGTAGTNAAKIAVGLGSDVTILDINA
ERLRELDDLFGDHVTTLMSNSYHIAECVRESDLVVGAVLIPGAKAPKLVT
EEMVRSMTPGSVLVDIAIDQGGIFETTDRVTTHDDPTYVKHGVVHYAVAN
MPGAVPRTSTFALTNVTIPYALQIANKGYRAACLDNPALLKGINTLDGHI
VYEAVAAAHNMPYTDVHSLLHG

In a first aspect of the present invention, there is provided a method of constructing a DL-alanine-producing genetically engineered strain, including the steps of:
providing an original strain possessing a pyruvate synthesis pathway;
constructing a genetically engineered strain for a two-step method by engineering the genome of the original strain through steps S200 and S300, or constructing a genetically engineered strain overexpressing an alanine racemase gene through steps S200, S300 and S400;
S200: introducing overexpressed 6-phosphofructokinase and pyruvate kinase genes;
S300: introducing an overexpressed gene encoding thermostable alanine dehydrogenase;
S400: inactivating or deleting (preferably, completely) an alanine racemase gene contained in the genome of the original strain and then introducing an overexpressed alanine racemase gene.

It will be understood that the genome of the original strain may contain and can express an alanine racemase gene. That is, the original strain possesses a D-alanine synthesis pathway.

In some embodiments of the present invention, the genome of the original strain contains an alanine racemase gene, and the method includes the steps of:
S200: inserting a copy of the pfk gene that encodes 6-phosphofructokinase and a copy of the pyk gene that encodes pyruvate kinase;
S300: inserting a gene encoding thermostable alanine dehydrogenase, preferably the GSald gene that encodes alanine dehydrogenase which is thermostable at 42° C. to 55° C.;
S400: inactivating or deleting (preferably, completely) the alanine racemase gene and then introducing an overexpressed alanine racemase gene.

A DL-alanine-producing genetically engineered strain constructed according to the first aspect of the present invention is capable of production of DL-alanine by fermentation under a high-temperature condition of 42° C. or higher (e.g., 42° C. to 55° C.). Step S200 can enhance the glycolysis pathway by enabling overexpression of 6-phosphofructokinase and pyruvate kinase involved in the pathway, thereby providing an increased supply of pyruvate and promoting the production of DL-alanine. In step S300, the gene encoding thermostable alanine dehydrogenase is introduced to enhance a pathway that synthesizes DL-alanine from pyruvate, effectively increasing the yield of DL-alanine. The genome engineering in step S400 enables high-yield production of optically pure DL-alanine with a simplified process at reduced cost. Combining these strategies can synergistically achieve high-yield production of racemic DL-alanine under a high-temperature condition.

In some embodiments of the present invention, the original strain further possesses a lactate synthesis pathway, and the genome of the original strain contains a lactate dehydrogenase gene. In these cases, the method further includes the step of:
S100: inactivating or deleting the lactate dehydrogenase gene in the genome of the original strain.

In this way, through S100, the lactate synthesis pathway of the original strain can be blocked to enhance the pyruvate synthesis pathway, thus increasing the yield of DL-alanine.

In some embodiments of the present invention, a lactate dehydrogenase gene in the original strain is inactivated or deleted, and genes encoding 6-phosphofructokinase and pyruvate kinase in the original strain are overexpressed. Moreover, an overexpressed alanine dehydrogenase gene is introduced, and an endogenous alanine racemase gene in the original strain is inactivated or deleted. Additionally, an overexpressed alanine racemase gene is introduced.

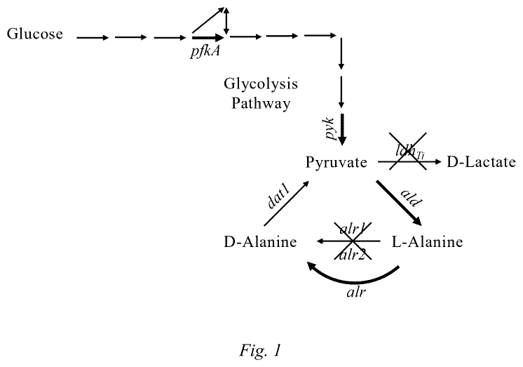

FIG. 1 schematically illustrates alanine synthesis and metabolism pathways in a DL-alanine-producing genetically engineered strain according to some embodiments of the present invention. A carbon source is converted into pyruvate through the glycolysis pathway, which then reacts with and consumes NADH and ammonium ions under the catalysis of alanine dehydrogenase, forming L-alanine. Part of the resulting L-alanine is directly secreted extracellularly, and the remainder is converted under the catalysis of alanine racemase into D-alanine, which is then secreted extracellularly. In case the carbon flux downstream of pyruvate is predominated by the lactate synthesis pathway in the original strain, blocking the lactate synthesis pathway can direct the carbon flux to the alanine synthesis pathway as much as possible. Moreover, overexpressing 6-phosphofructokinase and pyruvate kinase that are involved in the glycolysis pathway can enhance the glycolysis pathway and result in an increased supply of pyruvate, thereby promoting the production of DL-alanine. Completely knocking out the endogenous alanine racemase gene can block the native D-alanine synthesis pathway, which exhibits a low level of expression and is controlled by the expression regulation system in the strain, leading to a lower production of D-alanine than L-Alanine. Introducing the overexpressed gene alanine racemase can promote D-alanine synthesis and enhance its controllability, thus enabling equal yields of D-alanine and L-alanine, which result in the obtainment of DL-alanine.

In some embodiments of the present invention, the original strain further possesses a D-lactate synthesis pathway, and its genome contains the ldh_{Ti} gene that encodes D-lactate dehydrogenase. In these cases, the method further includes the step of inactivating or deleting the ldh_{Ti} gene that encodes D-lactate dehydrogenase in the genome of the original strain (S500). In some preferred embodiments of the present invention, step S500 includes: knocking out the ldh_{Ti} gene that encodes D-lactate dehydrogenase in the genome of the original strain.

In some embodiments of the present invention, in step 500, the ldh_{Ti} gene that encodes D-lactate dehydrogenase has a sequence as shown in SEQ ID NO. 43.

In some embodiments of the present invention, in step S200, the pfk gene that encodes 6-phosphofructokinase has a sequence as shown in SEQ ID NO. 41, and the pyk gene that encodes pyruvate kinase has a sequence as shown in SEQ ID NO. 42.

In some embodiments of the present invention, in step S300, the alanine dehydrogenase gene has a sequence as shown in SEQ ID NO. 1.

In some embodiments of the present invention, in steps S200 and S300, each of the relevant genes is inserted by adding a copy or copies (i.e., one or more copies) of the gene to the chromosome and ligating in series a promoter upstream thereof. In some embodiments of the present invention, the promoter is any one of Pₐₗₛ, Plac, Ptrc, Ptac, Pc, P43 and the like. In some embodiments of the present invention, the promoter is Pₐₗₛ. In some embodiments of the present invention, the promoter has a sequence as shown in SEQ ID NO. 4.

It will be understood that the alanine racemase gene inactivated or deleted in step S400 is an endogenous gene.

In some embodiments of the present invention, in step S400, the alanine racemase gene is preferably completely inactivated or deleted, for example, by complete knockout.

In some embodiments of the present invention, in step S400, one, two or more alanine racemase genes are inactivated or deleted. Preferably, at least one endogenous alanine racemase gene is completely inactivated or completely deleted.

In some embodiments of the present invention, in step S400, the alr1 and alr2 genes that encode endogenous alanine racemase are completely inactivated or completely deleted. Preferably, the alr1 gene that encodes endogenous alanine racemase has a sequence as shown in SEQ ID NO. 2, and the alr2 gene that encodes endogenous alanine racemase has a sequence as shown in SEQ ID NO. 3.

In some embodiments of the present invention, in step S400, the overexpressed alanine racemase gene is not limited to being achieved by any particular method, and possible methods may include, but are not limited to, insertion of a strong promoter, addition of a copy or copies of the gene, sequence optimization of the gene, etc.

In some embodiments of the present invention, in step S400, the overexpressed alanine racemase gene is achieved by insertion of a strong promoter and an alanine racemase gene.

In some preferred embodiments of the present invention, the strong promoter has a sequence as shown in SEQ ID NO. 4.

In some preferred embodiments of the present invention, the alr1 or/and alr2 genes that encode alanine racemase are inserted. Moreover, the alr1 gene that encodes alanine racemase has a sequence as shown in SEQ ID NO. 2, and the alr2 gene that encodes alanine racemase has a sequence as shown in SEQ ID NO. 3.

In some embodiments of the present invention, the engineering of the genome of the original strain includes steps S100 and S200. In this way, the lactate synthesis pathway can be blocked by inactivating or deleting the lactate dehydrogenase gene, and the glycolysis pathway can be enhanced through overexpression of 6-phosphofructokinase by the pfk gene and of pyruvate kinase by the pyk gene. Preferably, the engineering of the genome of the original strain further includes step S300. In this way, the yield of L-alanine can be additionally increased by inserting the gene that encodes thermostable alanine dehydrogenase.

In some embodiments of the present invention, the engineering of the genome of the original strain includes steps S100, S200, S300 and S400. In this way, in addition to blocking the lactate synthesis pathway by inactivating or deleting the lactate dehydrogenase gene and to enhancing the glycolysis pathway and thereby providing an increased supply of pyruvate through introducing the overexpressed 6-phosphofructokinase and pyruvate kinase genes to the original strain, the introduced overexpressed alanine dehydrogenase gene can enhance the synthesis pathway of L-alanine from pyruvate, and the endogenous alanine racemase gene in the original strain is inactivated or deleted, followed by introducing the overexpressed alanine racemase gene. As a result of these synergistic strategies in the steps, the resultant genetically engineered strain is capable of direct, high-yield production of DL-alanine by fermentation under a high-temperature condition.

In some embodiments of the present invention, the engineering of the genome of the original strain includes steps S100, S200, S300 and S400. In this way, through completely inactivating or completely deleting the endogenous alanine racemase gene in the original strain and introducing a desired number of copies of the alanine racemase gene, overexpression of alanine racemase can be controlled to achieve controllable high-yield production of racemic DL-alanine.

In some embodiments of the present invention, the engineering of the genome of the original strain includes steps S500 and S200. In this way, the lactate synthesis pathway is blocked by inactivating or deleting the ldh_{Ti} gene that encodes D-lactate dehydrogenase, and the glycolysis pathway is enhanced by overexpressing the pfk and pyk genes that encode 6-phosphofructokinase and pyruvate kinase, respectively. Preferably, the engineering of the genome of the original strain further includes step S300 in order to introduce the overexpressed gene encoding thermostable alanine dehydrogenase, which is preferred to be thermostable at 42° C. to 55° C.

In a preferred embodiment of the present invention, the method includes the steps of:
S500: completely inactivating or completely deleting a lactate dehydrogenase gene in the genome of the original strain, preferably the ldh_{Ti} gene that encodes D-lactate dehydrogenase (thereby blocking the lactate synthesis pathway);
S200: overexpressing a copy of the pfk gene that encodes 6-phosphofructokinase and the pyk gene that encodes pyruvate kinase (thereby enhancing the glycolysis pathway and providing an increased supply of pyruvate);
S300: overexpressing the GSald gene that encodes alanine dehydrogenase and is separately introduced (thereby enhancing the synthesis pathway from pyruvate to L-alanine); and
S400: completely inactivating or completely deleting the endogenous alr1 and alr2 genes that encode alanine racemase and then overexpressing the alr1 or/and alr2 genes that encode alanine racemase (thereby enabling controllable conversion of L-alanine to D-alanine and facilitating the production of racemic DL-alanine).

In some embodiments of the present invention, the genes are deleted by knockout.

In some embodiments of the present invention, in steps S200 and S300, each of the genes is overexpressed by adding a copy or copies (i.e., one or more copies) of the gene to the chromosome and ligating in series a promoter upstream thereof.

In a preferred embodiment of the present invention, the method includes the steps of:
S500: knocking out a lactate dehydrogenase gene in the genome of the original strain, preferably the ldh_{Ti} gene that encodes D-lactate dehydrogenase (thereby blocking the lactate synthesis pathway);
S200: inserting a copy of the pfk gene that encodes 6-phosphofructokinase and a copy of the pyk gene that encodes pyruvate kinase (thereby enabling overexpression of the pfk gene that encodes 6-phosphofructokinase and the pyk gene that encodes pyruvate kinase in the original strain, enhancing the glycolysis pathway and providing an increased supply of pyruvate);
S300: inserting the GSald gene that encodes alanine dehydrogenase and is from a heterologous source (thereby enabling overexpression of the heterologous GSald gene that encodes alanine dehydrogenase and enhancing the synthesis pathway from pyruvate to L-alanine); and
S400: knocking out the endogenous alr1 and alr2 genes that encode alanine racemase and then inserting the alr1 or/and alr2 genes that encode alanine racemase (thereby enabling controllable conversion of L-alanine to D-alanine and facilitating high-yield production of racemic DL-alanine).

In some embodiments of the present invention, the original strain is a thermophilic strain.

In some preferred embodiments of the present invention, the original strain is a Bacillus strain.

In some preferred embodiments of the present invention, the original strain is Bacillus licheniformis, which is a facultative anaerobic, Gram-positive, endospore-forming bacterium with a fast growth rate, a wide substrate spectrum and capabilities of high-temperature fermentation under a condition of 50° C. Moreover, it allows for stable genetic manipulation and has been recognized by the US Food and Drug Administration as a “generally regarded as safe” (GRAS) bacterium. Thus, it is promising for providing an ideal platform strain.

In some preferred embodiments of the present invention, the original strain is Bacillus licheniformis, Bacillus coagulans, Bacillus methylotrophicus, thermophilic Bacillus inulinus or Geobacillus stearothermophilus.

In some preferred embodiments of the present invention, the original strain is Bacillus licheniformis ATCC 14580 or a derivative thereof. Examples of the derivative include, but are not limited to, Bacillus licheniformis MW3, Bacillus licheniformis BN11, etc.

In some preferred embodiments of the present invention, the original strain is Bacillus licheniformis BN11, deposited in the China Center for Type Culture Collection on Jan. 8, 2016 as CCTCC NO: M2016026.

In some preferred embodiments of the present invention, the original strain is Bacillus licheniformis BN11, wherein the pfk gene that encodes 6-phosphofructokinase has a sequence as shown in SEQ ID NO. 41, and the pyk gene that encodes pyruvate kinase has a sequence as shown in SEQ ID NO. 42.

In some preferred embodiments of the present invention, the original strain is Bacillus licheniformis BN11, wherein the GSald gene that encodes alanine dehydrogenase has a sequence as shown in SEQ ID NO. 1.

In some preferred embodiments of the present invention, the original strain is Bacillus licheniformis BN11, wherein the endogenous alanine racemase gene includes the alr1 and alr2 genes, which have sequences as shown in SEQ ID NOs. 2 and 3, respectively.

In some preferred embodiments of the present invention, the separately introduced alanine racemase gene includes alr1 (SEQ ID NO. 2) or alr2 (SEQ ID NO. 3).

In some preferred embodiments of the present invention, the original strain is Bacillus licheniformis BN11, wherein the ldh_{Ti} gene that encodes D-lactate dehydrogenase has a sequence as shown in SEQ ID NO. 43.

It will be understood that, unless otherwise specified, the features of the embodiments of the present invention are preferred to be independent, but can be combined as appropriate.

In a second aspect of the present invention, there is provided a genetically engineered strain constructed in accordance with the first aspect of the present invention. The genetically engineered strain is capable of production of DL-alanine by fermentation at 42° C. or higher (e.g., 42° C. to 55° C.) with a significantly increased yield. It is also capable of high-yield production of racemic DL-alanine.

In a third aspect of the present invention, there is provided use of a genetically engineered strain according to the second aspect of the present invention for DL-alanine production. It can be used for production of DL-alanine by fermentation with a significantly increased yield under a high-temperature condition. It can be also used for high-yield production of racemic DL-alanine under a high-temperature condition.

In a fourth aspect of the present invention, there is provided a method of producing DL-alanine, in which a genetically engineered strain according to the second aspect of the present invention is subject to fermentation culture at a temperature of 42° C. to 55° C. The fermentation culture may be accomplished by any one selected from:
a two-step method using a genetically engineered strain for this method, which includes a fermentation culture step and an alanine racemase treatment step;
a direct fermentation method using a genetically engineered strain having an overexpressed alanine racemase gene, which includes a fermentation culture step and does not include an additional alanine racemase treatment step; and
a hybrid method using a genetically engineered strain having an overexpressed alanine racemase gene, which includes both a fermentation culture step and an alanine racemase treatment step and allows more flexible optical purity control.

It would be appreciated that optical purity control of alanine can be achieved by using a genetically engineered strain for the two-step method in combination with an additional alanine racemase treatment step. However, this would obviously require the use of more alanine racemase than the method using a genetically engineered strain having an overexpressed alanine racemase gene, leading to higher cost.

In some embodiments of the present invention, in the method, before the fermentation culture step, seed culture is carried out to result in activated seeds, which are then inoculated into fermentation culture medium, followed by the fermentation culture.

In some embodiments of the present invention, the seed culture step includes inoculating the genetically engineered strain into seed culture medium and culturing it under a condition at a temperature of 42° C. to 55° C., resulting in a seed culture solution, i.e., activated seeds.

In some embodiments of the present invention, the seed culture medium contains the following components: peptone, yeast powder and sodium chloride.

In some embodiments of the present invention, the seed culture medium is composed of the following components: peptone, yeast powder and sodium chloride.

Examples of the fermentation culture temperature include, but are not limited to, 42° C., 43° C., 44° C., 45° C., 46° C., 47° C., 48° C., 49° C., 50° C., 51° C., 52° C., 53° C., 54° C. and 55° C.

In some embodiments of the present invention, the fermentation culture step includes: inoculating the activated seeds into fermentation culture medium containing a carbon source and carrying out fed-batch fermentation under a condition at a fermentation culture temperature of 42° C. to 55° C. until a predetermined broth density is achieved. Moreover, an inoculum volume of the activated seeds, measured in percentage by volume, may be 3% to 5%, for example, such as 3%, 4% or 5%.

In some embodiments of the present invention, in the fermentation culture step, the carbon source is glucose, glycerol, xylose or arabinose.

In some embodiments of the present invention, in the fermentation culture step, the fermentation culture medium containsthe following components: glucose, yeast powder, ammonium sulfate, dipotassium hydrogen phosphate, potassium dihydrogen phosphate, magnesium sulfate, ferrous sulfate and manganese sulfate. In some embodiments of the present invention, the fermentation culture medium is composed ofthe following components: glucose, yeast powder, ammonium sulfate, dipotassium hydrogen phosphate, potassium dihydrogen phosphate, magnesium sulfate, ferrous sulfate and manganese sulfate.

In some embodiments of the present invention, the achievement of the predetermined broth density is determined based on a detected OD₆₀₀ value. For example, the fermentation is stopped when an OD₆₀₀ of 6.5-8.0 is detected.

In some embodiments of the present invention, the fermentation culture step includes: pH adjustment to about 7.0 with ammonia water; fermentation culture under aeration and agitation (first fermentation culture stage) until an OD₆₀₀ value of 6.5-8.0, followed by stopping aeration; and continued fermentation culture at a reduced agitation rate (second fermentation culture stage) until a predetermined broth density is attained, followed by stopping the fermentation. During the fermentation process, the carbon source may be supplemented when its concentration drops to a certain value.

The ammonia water for pH adjustment may have a concentration of 20-30 percent by volume (v/v), such as 20%, 25% or 30%.

Preferably, the aeration is accomplished with air.

The aeration may be conducted at an aeration rate of 0.8 vvm to 1.2 vvm, such as 0.9 vvm, 1.0 vvm, 1.1 vvm, 1.2 vvm or the like.

The fermentation culture is divided into multiple stages. For example, it may be divided into two stages, including a first fermentation culture stage with aeration and a second fermentation culture stage without aeration. The first fermentation culture stage allows fast growth of the strain, and the second fermentation culture stage enables efficient L-alanine production. The first fermentation culture stage is preferred to be with agitation at a speed of 280 rpm to 320 rpm, such as, for example, 280 rpm, 290 rpm, 300 rpm, 310 rpm, 320 rpm or the like. The first fermentation culture stage lasts for a period of time depending on an OD₆₀₀ value, for example, 6 hours to 8 hours, such as 6 h, 6.5 h, 7 h, 7.5 h, or 8 h. The second fermentation culture stage is preferred to be with agitation at a speed of 60 rpm to 100 rpm, such as, for example, 60 rpm, 70 rpm, 80 rpm, 90 rpm, 100 rpm or the like. The second fermentation culture stage lasts for a period of time also depending on an OD₆₀₀ value (e.g., 1.2), for example, 50 hours to 70 hours, such as 50 h, 55 h, 60 h, 65 h or 70 h.

In the fermentation process, the carbon source may be supplemented once, twice or more times.

Regarding the timing of carbon source supplementation during the fermentation process, it may be supplemented, for example, once its concentration drops to 30 g/L to 50 g/L, and again supplemented, for example, when the concentration drops to 20 g/L to 40 g/L. Preferably, the carbon source is glucose.

In some embodiments of the present invention, the carbon source is supplemented only once. In these cases, the carbon source may be supplemented upon the concentration dropping to 30 g/L to 50 g/L (e.g., 30 g/L, 35 g/L, 40 g/L, 45 g/L or 50 g/L), thereby increasing the concentration to 80 g/L to 120 g/L (e.g., 80 g/L, 90 g/L, 100 g/L, 110 g/L or 120 g/L). Preferably, the carbon source is glucose.

In some embodiments of the present invention, the carbon source is supplemented twice: the first supplementation may occur when the concentration drops to 30 g/L to 50 g/L (e.g., 30 g/L, 35 g/L, 40 g/L, 45 g/L or 50 g/L), resulting in a new concentration of 80 g/L to 120 g/L (e.g., 80 g/L, 90 g/L, 100 g/L, 110 g/L or 120 g/L); and the second supplementation may be conducted upon the concentration dropping to 20 g/L to 40 g/L (e.g., 20 g/L, 25 g/L, 30 g/L, 35 g/L or 40 g/L) to raise the concentration back to 60 g/L to 80 g/L (e.g., 60 g/L, 65 g/L, 70 g/L, 75 g/L or 80 g/L). Preferably, the carbon source is glucose.

In some embodiments of the present invention, the fermentation culture step includes: pH adjustment to 7.0±0.2 with 20-30% (v/v) ammonia water; fermentation culture for 6-8 hours under aeration at a rate of 0.8 vvm to 1.2 vvm and under agitation at an initial speed of 280 rpm to 320 rpm so that an OD₆₀₀ value of 6.5-8.0 is achieved, followed by stopping aeration; and continued fermentation culture for 50 hours to 70 hours (e.g., 60 hours) at a modified agitation speed of 60 rpm to 100 rpm, followed by stopping the fermentation. Moreover, during the fermentation process, the carbon source may be supplemented once, twice or more times when its concentration drops to a certain value. In some embodiments, the carbon source is supplemented only once. In these cases, the carbon source may be supplemented upon the concentration dropping to 30 g/L to 50 g/L, increasing the concentration to 80 g/L to 120 g/L. In some other embodiments, the carbon source is supplemented twice. In these cases, the carbon source is supplemented for the first time when its concentration drops to 30 g/L to 50 g/L so that the concentration rises back to 80 g/L to 120 g/L. Subsequently, when the concentration further drops to 20 g/L to 40 g/L, the carbon source is supplemented for a second time to raise the concentration back to 60 g/L to 80 g/L. Preferably, the carbon source is glucose.

In some embodiments of the present invention, the carbon source in the fermentation culture step is glucose, and the fermentation culture step includes: pH adjustment and maintenance to and at 7.0 with 25% ammonia water; fermentation culture for 6 hours to 8 hours under aeration at a rate of 1 vvm and under agitation at an initial speed of 300 rpm so that an OD₆₀₀ value of the Bacillus licheniformis strain reaches 6.5-8.0, followed by stopping aeration; and continued fermentation culture for 60 h at a modified agitation speed of 80 rpm, followed by stopping the fermentation. During the fermentation process, glucose is supplemented when its concentration drops to 30 g/L to 50 g/L, thereby increasing the glucose concentration to 100 g/L. In some implementations, when the glucose concentration drops to 20-40 g/L at a later time, glucose supplementation is again conducted to raise the glucose concentration to 70 g/L.

In some embodiments of the present invention, in the alanine racemase treatment step, alanine racemase used is provided by purified alanine racemase or/and a bacterium overexpressing an encoding sequence for alanine racemase. The bacterium that overexpresses the encoding sequence for alanine racemase may provide desired alanine racemase. The host bacterium is not limited to any particular type, as long as it does not interfere with the DL-alanine synthesis pathway. Examples include, but are not limited to, Escherichia coli and the like.

The racemase treatment is preferably carried out at a high temperature (e.g., 42° C. to 55° C.). From point of view of processing, simplified operation can be achieved when the treatment temperature is equal to the fermentation temperature. Examples of the temperature for the racemase treatment include, but are not limited to, 42° C., 43° C., 44° C., 45° C., 46° C., 47° C., 48° C., 49° C., 50° C., 51° C., 52° C., 53° C., 54° C., 55° C., etc.

Desirably, the racemase treatment is conducted until there is no further increase in optical purity. Preferably, it is conducted until the optical purity of the L-alanine reaches 50%. For example, it may last for 12 h to 24h, such as 12 h, 16 h, 18 h, 24 h or the like.

In some embodiments of the present invention, the alanine racemase treatment step includes: at the end of the fermentation process, directly adding an appropriate amount of alanine racemase to the fermentation broth and leaving it at 50° C. for 12 h. In addition, the added alanine racemase is provided by an Escherichia coli strain that overexpresses the racemase gene.

The present invention will be described in greater detail below by way of a few examples, which are intended to be illustrative rather than limiting the scope of the present invention in any sense. These examples are provided in accordance with the teachings of the present invention. Although specific implementation and operation details are set forth, the scope of the invention is in no way limited to the following examples. For any experimental procedure employed in the following examples (e.g., PCR amplification, transformation, gene knockout, gene insertion, etc.), if no particular conditions are specified, it is preferably carried out in the manner and conditions described above. Otherwise, it is generally carried out in a conventional manner and conditions, for example, as taught in Molecular Cloning: A Laboratory Manual by Sambrook et al. (New York: Cold Spring Harbor Laboratory Press, 1989), or under conditions suggested by the manufacturer.

All the materials and reagents used in the following examples are available from commercial sources, unless otherwise specified.

An alanine concentration in each fermentation broth sample was measured by high-performance liquid chromatography (HPLC). Sodium 1-octanesulfonate was weighed at 0.54 g and added to 800 mL of ultrapure water, and the pH was then adjusted to 2.1 with phosphoric acid. Methanol at 100 mL and ultrapure water were then successively added so that a volume of 1 L was obtained, as a mobile phase. The sample was injected at a volume of 10 μL into a ZORBAX Eclipse XDB-C18 column (150 mm×4.6 mm, 5 μm) at a temperature of 30° C. and caused to flow through the column at a flow rate of 0.8 mL/min, and the measurement was performed by a DAD detector at a wavelength of 210 nm. Before being measured, the sample was boiled for 10 min and centrifuged at 8000 rpm for 5 min. The supernatant was then taken and diluted an appropriate number of times so that alanine was present therein at a final concentration of 0.2 g/L to 1 g/L.

Optical purity of alanine in each fermentation broth sample was measured by HPLC using a 2 mM aqueous copper sulfate solution as a mobile phase. The sample was injected at a volume of 10 μL into a SUMICHIRAL OA-5000 (150 mm×4.6 mm, 5 μm) at a temperature of 30° C. and caused to flow through the column at a flow rate of 0.5 mL/min, and the measurement was performed by a DAD detector at a wavelength of 254 nm. Before the measurement, the sample was boiled for 10 min and centrifuged at 8000 rpm for 5 min. The supernatant was then taken and diluted an appropriate number of times so that alanine was present therein at a final concentration of 0.2 g/L to 1 g/L.

Glucose concentrations were measured on an SBA-40D biosensor analyzer (Biology Institute of Shandong Academy of Sciences).

In the following examples, Bacillus licheniformis strains were used as original strains to construct genetically engineered strains capable of producing DL-alanine under a high-temperature condition. It would be appreciated that, in the following examples, strains of other bacteria (for example, including, but not limited to, Bacillus coagulans, Bacillus methylotrophicus, thermophilic Bacillus inulinus, Geobacillus stearothermophilus, etc.) may also be used as original strains to construct genetically engineered strains capable of producing DL-alanine under a high-temperature condition, without departing from the scope of the present invention.

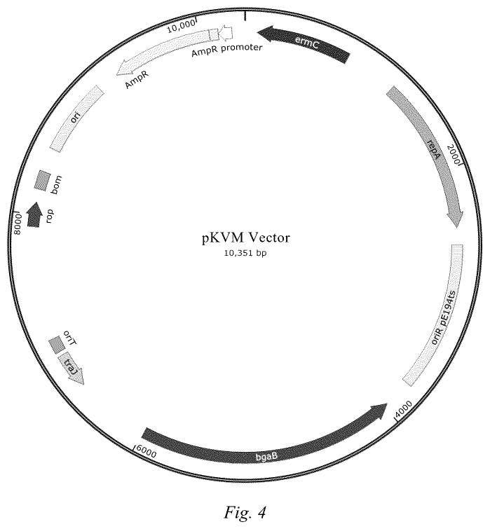

FIG. 4 shows the structure of a pKVM vector, which contains two antibiotic resistance genes: ampR (ampicillin resistance) and ermC (erythromycin resistance), as well as the bgaB gene that encodes galactosidase. All of these genes can be used as selectable markers. This plasmid has a heat-sensitive origin of replication (oriT pE194ts) and origins of replication in Escherichia coli (ori) and Bacillus licheniformis (oriT).

Bacillus licheniformis BN11 is a lactate-producing strain constructed from ATCC 14580. It was deposited in the China Center for Type Culture Collection on Jan. 8, 2016 as CCTCC NO: M2016026.

Inoculum volumes are measured in percentage by volume.

Unless otherwise specified, for any newly inserted gene, one copy of it was inserted.

### EXAMPLE 1: CONSTRUCTION OF BACILLUS LICHENIFORMIS STRAIN WITH D-LACTATE SYNTHESIS PATHWAY BEING KNOCKED OUT

1.1 Primers were designed according to the sequence of the Bacillus licheniformis ATCC 14580 genome (GenBank No. NC_006270.3). Separately with pKVM-XPDdldh-UpF (SEQ ID NO. 5) and XPDdldh-UpR (SEQ ID NO. 6), as well as, XPDdldh-DownF (SEQ ID NO. 7) and pKVM-XPDdldh-DownR (SEQ ID NO. 8), as upstream and downstream primers, respectively, PCR amplification was performed to obtain fragments containing homology arms upstream and downstream of a promoter for the gene ldh_{Ti}.

1.2 The PCR product from step 1.1 was purified, and recombinant PCR amplification was conducted with pKVM-XPDdldh-UpF and pKVM-XPDdldh-DownR as primers. After being purified, the PCR product was cloned into a pKVM vector by seamless cloning, which was then transformed into Escherichia coli S17, resulting in S17-pKVMΔldh_{Ti}.

1.3 Strains of S17-pKVMΔldh_{Ti} (Escherichia coli) obtained in step 1.2 and Bacillus licheniformis BN11 (original strain) were separately cultured by fermentation to an OD₆₀₀ value of 1.2. The resulting strains were then centrifuged at 6000 rpm for 5 min and washed twice with PBS. Subsequently, the engineered Escherichia coli S17-pKVMΔldh_{Ti} and the Bacillus licheniformis BN11 strains with the same OD₆₀₀ (and hence the same concentration) were mixed up at a volume ratio of 7:1, and the mixture was added dropwise to LB plates. After overnight culture at 30° C., the strains were diluted and spread on LB plates containing erythromycin and polymyxin, followed by culture at 30° C. The screened transformants were cultured at 37° C. in LB medium containing erythromycin and then transferred to and cultured in LB medium at 50° C. After that, they are diluted, spread on LB plates containing erythromycin and cultured at 50° C., in order to screen for single-crossover transformants. The single-crossover transformants were continuously cultured in LB medium at 30° C. for two generations, diluted, spread on LB plates and cultured at 37° C. overnight. Double-crossover transformants were then selected by reverse screening based on resistance to erythromycin. As verified by PCR using pKVM-XPDdldh-UpF and pKVM-XPDdldh-DownR as primers and sequencing, a genetically engineered strain with a D-lactate synthesis pathway (i.e., the gene ldh_{Ti} encoding D-lactate dehydrogenase (SEQ ID NO. 43)) being knocked out was successfully constructed, denoted as BN11Δldh_{Ti}.

### EXAMPLE 2: CONSTRUCTION OF BACILLUS LICHENIFORMIS STRAIN OVEREXPRESSING 6-PHOSPHOFRUCTOKINASE AND PYRUVATE KINASE

2.1 Primers were designed according to the sequence of the Bacillus licheniformis ATCC 14580 genome (GenBank No. NC_006270.3). Separately with pKVM-XPDldh-UpF (SEQ ID NO. 9) and Pₐₗₛ-XPDldh-UpR (SEQ ID NO. 10), XPDldh-Pₐₗₛ-F (SEQ ID NO. 11) and PFYAK-Pₐₗₛ-R (SEQ ID NO. 12), Pₐₗₛ-PFYAK-F (SEQ ID NO. 13) and XPDldh-PFYAK-R (SEQ ID NO. 14), as well as PFYAK-XPDldh-DownF (SEQ ID NO. 15) and pKVM-XPDldh-DownR (SEQ ID NO. 16), as upstream and downstream primers, respectively, PCR amplification was performed to obtain fragments containing a homology arm upstream of the ldh gene, the Pₐₗₛ promoter (SEQ ID NO. 4), the pfk (SEQ ID NO. 41) and pyk (SEQ ID NO. 42) gene clusters and a homology arm downstream of the ldh gene.

2.2 The PCR product from step 2.1 was purified, and recombinant PCR amplification was conducted with pKVM-XPDldh-UpF and pKVM-XPDldh-DownR as primers. After being purified, the PCR product was cloned into a pKVM vector by seamless cloning, which was then transformed into Escherichia coli S17, resulting in S17-pKVM-PFYAK.

2.3 Strains of Escherichia coli S17-pKVM-PFYAK obtained in step 2.2 and Bacillus licheniformis BN11Δldh_{Ti} were subjected to conjugation transfer and homologous recombination conducted in the same manner as in Example 1, resulting in a strain overexpressing the pfk and pyk genes, denoted as BN11Δldh_{Ti}-PFYAK.

### EXAMPLE 3: CONSTRUCTION OF STRAIN OVEREXPRESSING HETEROLOGOUS ALANINE DEHYDROGENASE

3.1 Based on the amino acid sequence of alanine dehydrogenase, the BLAST method was used to identify potentially thermostable alanine dehydrogenase (with an amino acid sequence as shown in SEQ ID NO. 44 and a protein sequence number corresponding to WP_033014465.1) in the genome of Geobacillus stearothermophilus. This alanine dehydrogenase gene sourced from Geobacillus stearothermophilus, named GSald, was subject to codon optimization based on the Bacillus licheniformis genome (a sequence of the optimized version is as shown in SEQ ID NO. 1) and synthesized together with the Pₐₗₛ promoter (SEQ ID NO. 4) from 5′ to 3′. The merged sequence is denoted as Pₐₗₛ-GSald.

3.2 Primers were designed according to the sequence of the Bacillus licheniformis ATCC 14580 genome (GenBank No. NC_006270.3). Separately with pKVM-XPDdldh-UpF (SEQ ID NO. 5) and Pₐₗₛ-XPDdldh-UpR (SEQ ID NO. 17), XPDdldh-Pₐₗₛ-F (SEQ ID NO. 18) and XPDdldh-GSald-R (SEQ ID NO. 19), as well as GSald-XPDdldh-DownF (SEQ ID NO. 20) and pKVM-XPDdldh-DownR (SEQ ID NO. 8), as upstream and downstream primers, respectively, PCR amplification was conducted to obtain fragments containing a homology arm upstream of a promoter for the ldh gene, the Pₐₗₛ-GSald gene and a homology arm downstream of the ldh_{Ti} gene.

3.3 The PCR product from step 3.2 was purified, and recombinant PCR amplification was conducted with pKVM-XPDdldh-UpF and pKVM-XPDdldh-DownR as primers. After being purified, the PCR product was cloned into a pKVM vector by seamless cloning, which was then transformed into Escherichia coli S17, resulting in S17-pKVM-GSald. Strains of Escherichia coli S17-pKVM-GSald and Bacillus licheniformis BN11AldhTi-PFYAK were subjected to conjugation transfer and homologous recombination conducted in the same manner as in Example 1, resulting in a strain expressing heterologous GSald, denoted as BA-1.

### EXAMPLE 4: KNOCKOUT OF ALANINE RACEMASE GENES IN BACILLUS LICHENIFORMIS

4.1 Primers were designed according to the sequence of the Bacillus licheniformis ATCC 14580 genome (GenBank No. NC_006270.3). Separately with pKVM-XPDalr1-UpF (SEQ ID NO. 21) and XPDalr1-UpR (SEQ ID NO. 22), as well as, XPDalr1-DownF (SEQ ID NO. 23) and pKVM-XPDalr1-DownR (SEQ ID NO. 24), as upstream and downstream primers, respectively, PCR amplification was performed to obtain fragments containing homology arms upstream and downstream of the gene alr1.

4.2 The PCR product from step 4.1 was purified, and recombinant PCR amplification was conducted with pKVM-XPDalr1-UpF and pKVM-XPDalr1-DownR as primers. After being purified, the PCR product was cloned into a pKVM vector by seamless cloning, which was then transformed into Escherichia coli S17, resulting in S17-pKVMΔalr1.

4.3 Strains of Escherichia coli S17-pKVMΔalr1 and Bacillus licheniformis BA-1 were subjected to conjugation transfer and homologous recombination conducted in the same manner as in Example 1, resulting in a strain with the alr1 gene being knocked out, denoted as BA-1Δalr1.

4.4. Primers were designed according to the sequence of the Bacillus licheniformis ATCC 14580 genome (GenBank No. NC_006270.3). Separately with pKVM-XPDalr2-UpF (SEQ ID NO. 25) and XPDalr2-UpR (SEQ ID NO. 26), as well as, XPDalr2-DownF (SEQ ID NO. 27) and pKVM-XPDalr2-DownR (SEQ ID NO. 28), as upstream and downstream primers, respectively, PCR amplification was performed to obtain fragments containing homology arms upstream and downstream of the gene alr2.

4.5 The PCR product from step 4.4 was purified, and recombinant PCR amplification was conducted with pKVM-XPDalr2-UpF and pKVM-XPDalr2-DownR as primers. After being purified, the PCR product was cloned into a pKVM vector by seamless cloning, which was then transformed into Escherichia coli S17, resulting in S17-pKVMΔalr2.

4.6 Strains of Escherichia coli S17-pKVMΔalr2 and Bacillus licheniformis BA-1Δalr1 were subjected to conjugation transfer and homologous recombination conducted in the same manner as in Example 1, resulting in a strain with the alr1 and alr2 genes being both knocked out, denoted as BLA-1.

### EXAMPLE 5: CONSTRUCTION OF STRAINS OVEREXPRESSING ALANINE RACEMASE

5.1 In the genome of Bacillus licheniformis ATCC 14580 (GenBank No. NC_006270.3), alanine racemase genes were identified and name respectively as alr1 (SEQ ID NO. 2) and alr2 (SEQ ID NO. 3). Primers were designed according to the sequence of the Bacillus licheniformis ATCC 14580 (GenBank No. NC_006270.3). Separately with pKVM-XPDPdh-UpF (SEQ ID NO. 29) and Pals-XPDPdh-UpR (SEQ ID NO. 30), XPDPdhUp-Pals-F (SEQ ID NO. 31) and Alr1-Pals-R (SEQ ID NO. 32), Pals-alr1-F (SEQ ID NO. 33) and XPDPdhDown-alr1-R (SEQ ID NO. 34), as well as Alr1-XPDPdh-DownF (SEQ ID NO. 35) and pKVM-XPDPdh-DownR (SEQ ID NO. 36), as upstream and downstream primers, respectively, PCR amplification was conducted to obtain fragments containing a homology arm upstream of the pdh gene, the Pₐₗₛ promoter, the alr1 gene and a homology arm downstream of the pdh gene.

5.2 The PCR product from step 5.1 was purified, and recombinant PCR amplification was conducted with pKVM-XPDPdh-UpF and pKVM-XPDPdh-DownR as primers. After being purified, the PCR product was cloned into a pKVM vector by seamless cloning, which was then transformed into Escherichia coli S17, resulting in S17-pKVM-Palr1. Strains of Escherichia coli S17-pKVM-Palr1 and Bacillus licheniformis BLA-1 were subjected to conjugation transfer and homologous recombination conducted in the same manner as in Example 1, resulting in a strain expressing alr1, denoted as BDLA-1.

5.3 Primers were designed according to the sequence of the Bacillus licheniformis ATCC 14580 (GenBank No. NC_006270.3). Separately with pKVM-XPDPdh-UpF (SEQ ID NO. 29) and Pals-XPDPdh-UpR (SEQ ID NO. 30), XPDPdhUp-Pals-F (SEQ ID NO. 31) and Alr2-Pals-R (SEQ ID NO. 37), Pals-alr2-F (SEQ ID NO. 38) and XPDPdhDown-alr2-R (SEQ ID NO. 39), as well as Alr2-XPDPdh-DownF (SEQ ID NO. 40) and pKVM-XPDPdh-DownR (SEQ ID NO. 36), as upstream and downstream primers, respectively, PCR amplification was conducted to obtain fragments containing a homology arm upstream of the pdh gene, the Pₐₗₛ promoter, the alr2 gene and a homology arm downstream of the pdh gene.

5.4 The PCR product from step 5.3 was purified, and recombinant PCR amplification was conducted with pKVM-XPDPdh-UpF and pKVM-XPDPdh-DownR as primers. After being purified, the PCR product was cloned into a pKVM vector by seamless cloning, which was then transformed into Escherichia coli S17, resulting in S17-pKVM-Palr2. Strains of Escherichia coli S17-pKVM-Palr2 and Bacillus licheniformis BLA-1 were subjected to conjugation transfer and homologous recombination conducted in the same manner as in Example 1, resulting in a strain expressing alr2, denoted as BDLA-2.

### EXAMPLE 6: FED-BATCH FERMENTATION (IN 5-L FERMENTER) OF RECOMBINANT BACILLUS LICHENIFORMIS BDLA-1 AT 42° C.

### 6.1 Seed Culture Medium and Fermentation Culture Medium

Seed culture medium composition: LB medium, yeast powder 5 g/L, tryptone 10 g/L, sodium chloride 10 g/L.

Fermentation culture medium composition: glucose 100 g/L, yeast powder 5 g/L, ammonium sulfate 5 g/L, dipotassium hydrogen phosphate trihydrate 1.3 g/L, potassium dihydrogen phosphate 0.5 g/L, magnesium sulfate heptahydrate 0.5 g/L, ferrous sulfate heptahydrate 20m g/L, manganese sulfate tetrahydrate 20m g/L. Glucose was separately sterilized at 115° C. for 20 min.

### 6.2 Seed Culture

BDLA-1 was transferred from a glycerol tube to a shake tube containing 5 mL of LB medium and incubated at 50° C. for 12 h with shaking at 200 rpm. After that, it was inoculated at an inoculum volume of 3% to 5% into 150 mL of LB medium contained in a 500-mL Erlenmeyer flask and incubated at 50° C. for 12 h with shaking at 200 rpm, resulting in a seed solution (i.e., activated seeds). In this step, seed activation was accomplished.

### 6.3 Fermentation Culture

The seed solution obtained in step 6.2 was inoculated at an inoculum volume of 5% into fermentation culture medium contained in a 5-L fermenter, resulting in a total volume of 3 L. After a fermentation pH was controlled to 7.0 with 25% (v/v) ammonia water, fermentation culture was started at a temperature of 42° C. under aeration (by air) at an aeration rate of 1.0 vvm and agitation at a speed of 300 rpm. After 6-8 hours, Bacillus licheniformis was fermented to an OD₆₀₀ of 6.5-8.0. At this time, the aeration was stopped, and the agitation speed was decreased to 80 rpm. The fermentation was then resumed and ended 60 h later. During the fermentation process, when a concentration of glucose dropped to 30 g/L to 50 g/L, glucose was supplemented to increase the concentration to 100 g/L. After that, upon the glucose concentration dropping to 20 g/L to 40 g/L, glucose was again supplemented to increase the concentration to 70 g/L. After 60 hours of fermentation, a DL-alanine concentration in the fermentation broth was detected as 104.7 g/L, corresponding to a glucose-to-alanine conversion rate of 68.6%, and the optical purity of L-alanine was 50.2%.

### EXAMPLE 7: FED-BATCH FERMENTATION (IN 5-L FERMENTER) OF RECOMBINANT BACILLUS LICHENIFORMIS BDLA-1 AT 50° C.

### 7.1 Seed Culture Medium and Fermentation Culture Medium

See 6.1 in Example 6

### 7.2. Seed Culture

See 6.2 in Example 6

### 7.3 Fermentation Culture

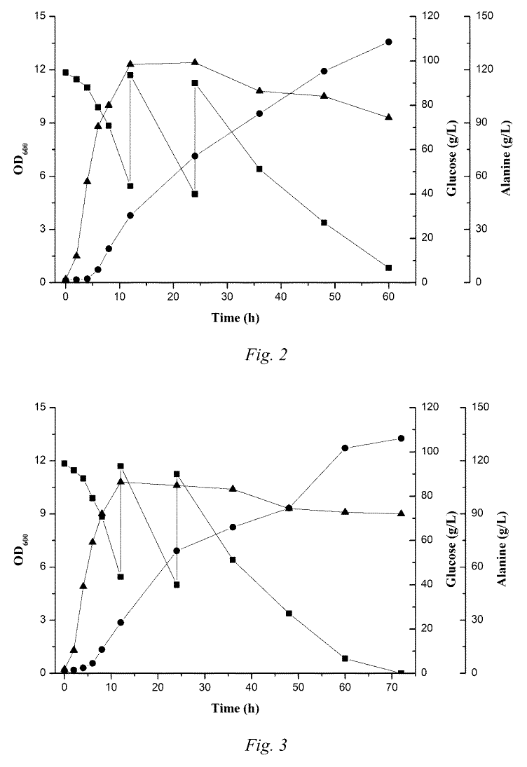

A seed solution obtained in step 7.2 was inoculated at an inoculum volume of 5% into fermentation culture medium contained in a 5-L fermenter, resulting in a total volume of 3 L. After a fermentation pH was controlled to 7.0 with 25% (v/v) ammonia water, fermentation culture was started at a temperature of 50° C. under aeration at an aeration rate of 1.0 vvm and agitation at a speed of 300 rpm. After 6-8 hours, Bacillus licheniformis was fermented to an OD₆₀₀ of 6.5-8.0. At this time, the aeration was stopped, and the agitation speed was decreased to 80 rpm. The fermentation was then resumed and ended 60 h later. During the fermentation process, when a concentration of glucose dropped to 30 g/L to 50 g/L, glucose was supplemented to increase the concentration to 100 g/L. FIG. 2 is a plot diagram of this fermentation process. After 60 hours of fermentation, a DL-alanine concentration in the fermentation broth was detected as 135.6 g/L, corresponding to a glucose-to-alanine conversion rate of 77.6%, and the optical purity of L-alanine was 49.7%.

### EXAMPLE 8: FED-BATCH FERMENTATION (IN 5-L FERMENTER) OF RECOMBINANT BACILLUS LICHENIFORMIS BDLA-1 AT 55° C.

### 8.1 Seed Culture Medium and Fermentation Culture Medium

See 6.1 in Example 6

### 8.2. Seed Culture

See 6.2 in Example 6

### 8.3 Fermentation Culture

A seed solution obtained in step 8.2 was inoculated at an inoculum volume of 5% into fermentation culture medium contained in a 5-L fermenter, resulting in a total volume of 3 L. After a fermentation pH was controlled to 7.0 with 25% (v/v) ammonia water, fermentation culture was started at a temperature of 55° C. under aeration at an aeration rate of 1.0 vvm and agitation at a speed of 300 rpm. After 6-8 hours, Bacillus licheniformis was fermented to an OD₆₀₀ of 3.0-4.5. At this time, the aeration was stopped, and the agitation speed was decreased to 80 rpm. The fermentation was then resumed and ended 60 h later. After 60 hours of fermentation, a DL-alanine concentration in the fermentation broth was detected as 18.9 g/L, corresponding to a glucose-to-alanine conversion rate of 31.5%, and the optical purity of L-alanine was 49.3%.

### EXAMPLE 9: FED-BATCH FERMENTATION (IN 50-L FERMENTER) OF RECOMBINANT BACILLUS LICHENIFORMIS BDLA-1 AT 42° C.

### 9.1 Seed Culture Medium and Fermentation Culture Medium

See 6.1 in Example 6

### 9.2 Seed Culture

BDLA-1 was transferred from a glycerol tube to a shake tube containing 5 mL of LB medium and incubated at 50° C. for 12 h with shaking at 200 rpm. After that, it was inoculated at an inoculum volume of 3% to 5% into 150 mL of LB medium contained in a 500-mL Erlenmeyer flask and incubated at 50° C. for 12 h with shaking at 200 rpm. Finally, it was further inoculated at an inoculum volume of 3% to 5% into 1.5 L of LB medium contained in a 5-L Erlenmeyer flask and incubated at 50° C. for 12 h with shaking at 200 rpm.

### 9.3 Fermentation Culture

A seed solution obtained in step 9.2 was inoculated at an inoculum volume of 5% into fermentation culture medium contained in a 50-L fermenter, resulting in a total volume of 30 L. After a fermentation pH was controlled to 7.0 with 25% (v/v) ammonia water, fermentation culture was started at a temperature of 42° C. under aeration at an aeration rate of 1.0 vvm and agitation at a speed of 300 rpm. After 6-8 hours, Bacillus licheniformis was fermented to an OD₆₀₀ of 6.5-8.0. At this time, the aeration was stopped, and the agitation speed was decreased to 80 rpm. The fermentation was then resumed and ended 60 h later. During the fermentation process, when a concentration of glucose dropped to 30 g/L to 50 g/L, glucose was supplemented to increase the concentration to 100 g/L. After 60 hours of fermentation, a DL-alanine concentration in the fermentation broth was detected as 53.3 g/L, corresponding to a glucose-to-alanine conversion rate of 65.0%, and the optical purity of L-alanine was 48.8%.

### EXAMPLE 10: FED-BATCH FERMENTATION (IN 50-L FERMENTER) OF RECOMBINANT BACILLUS LICHENIFORMIS BDLA-1 AT 50° C.

### 10.1 Seed Culture Medium and Fermentation Culture Medium

See 6.1 in Example 6

### 10.2 Seed Culture

See 9.2 in Example 9

### 10.3 Fermentation Culture

A seed solution obtained in step 10.2 was inoculated at an inoculum volume of 5% into fermentation culture medium contained in a 50-L fermenter, resulting in a total volume of 30 L. After a fermentation pH was controlled to 7.0 with 25% (v/v) ammonia water, fermentation culture was started at a temperature of 50° C. under aeration at an aeration rate of 1.0 vvm and agitation at a speed of 300 rpm. After 6-8 hours, Bacillus licheniformis was fermented to an OD₆₀₀ of 6.5-8.0. At this time, the aeration was stopped, and the agitation speed was decreased to 80 rpm. The fermentation was then resumed and ended 60 h later. During the fermentation process, when a concentration of glucose dropped to 30 g/L to 50 g/L, glucose was supplemented to increase the concentration to 100 g/L. After that, upon the glucose concentration further dropping to 20 g/L to 40 g/L, glucose was supplemented again to increase the concentration to 70 g/L. After 60 hours of fermentation, a DL-alanine concentration in the fermentation broth was detected as 126.3 g/L, corresponding to a glucose-to-alanine conversion rate of 78.0%, and the optical purity of L-alanine was 50.2%.

### EXAMPLE 11: FED-BATCH FERMENTATION (IN 50-L FERMENTER) OF RECOMBINANT BACILLUS LICHENIFORMIS BDLA-1 AT 55° C.

### 11.1 Seed Culture Medium and Fermentation Culture Medium

See 6.1 in Example 6

### 11.2 Seed Culture

See 9.2 in Example 9

### 11.3 Fermentation Culture

A seed solution obtained in step 11.2 was inoculated at an inoculum volume of 5% into fermentation culture medium contained in a 50-L fermenter, resulting in a total volume of 30 L. After a fermentation pH was controlled to 7.0 with 25% (v/v) ammonia water, fermentation culture was started at a temperature of 55° C. under aeration at an aeration rate of 1.0 vvm and agitation at a speed of 300 rpm. After 6-8 hours, Bacillus licheniformis was fermented to an OD₆₀₀ of 4.0-6.0. At this time, the aeration was stopped, and the agitation speed was decreased to 80 rpm. The fermentation was then resumed and ended 60 h later. After 60 hours of fermentation, a DL-alanine concentration in the fermentation broth was detected as 24.7 g/L, corresponding to a glucose-to-alanine conversion rate of 24.7%, and the optical purity of L-alanine was 24.7%.

### EXAMPLE 12: FED-BATCH FERMENTATION (IN 5-L FERMENTER) OF RECOMBINANT BACILLUS LICHENIFORMIS BDLA-2 AT 50° C.

### 12.1 Seed Culture Medium and Fermentation Culture Medium

See 6.1 in Example 6

### 12.2 Seed Culture

BDLA-2 was transferred from a glycerol tube to a shake tube containing 5 mL of LB medium and incubated at 50° C. for 12 h with shaking at 200 rpm. After that, it was inoculated at an inoculum volume of 3% to 5% into 150 mL of LB medium contained in a 500-mL Erlenmeyer flask and incubated at 50° C. for 12 h with shaking at 200 rpm, resulting in a seed solution.

### 12.3 Fermentation Culture

The seed solution obtained in step 12.2 was inoculated at an inoculum volume of 5% into fermentation culture medium contained in a 5-L fermenter, resulting in a total volume of 3 L. After a fermentation pH was controlled to 7.0 with 25% (v/v) ammonia water, fermentation culture was started at a temperature of 50° C. under aeration at an aeration rate of 1.0 vvm and agitation at a speed of 300 rpm. After 6-8 hours, Bacillus licheniformis was fermented to an OD₆₀₀ of 6.5-8.0. At this time, the aeration was stopped, and the agitation speed was decreased to 80 rpm. The fermentation was then resumed and ended 60 h later. During the fermentation process, when a concentration of glucose dropped to 30 g/L to 50 g/L, glucose was supplemented to increase the concentration to 100 g/L. After that, upon the glucose concentration dropping to 20 g/L to 40 g/L, glucose was again supplemented to increase the concentration to 70 g/L. After 60 hours of fermentation, a DL-alanine concentration in the fermentation broth was detected as 109.0 g/L, corresponding to a glucose-to-alanine conversion rate of 76.1%, and the optical purity of L-alanine was 53.0%.

### EXAMPLE 13: PRODUCTION (IN 5-L FERMENTER) OF DL-ALANINE BY RECOMBINANT BACILLUS LICHENIFORMIS BA-1 AT 50° C. USING TWO-STEP METHOD

### 13.1 Seed Culture Medium and Fermentation Culture Medium

See 6.1 in Example 6

### 13.2 Seed Culture

BA-1 was transferred from a glycerol tube to a shake tube containing 5 mL of LB medium and incubated at 50° C. for 12 h with shaking at 200 rpm. After that, it was inoculated at an inoculum volume of 3% to 5% into 150 mL of LB medium contained in a 500-mL Erlenmeyer flask and incubated at 50° C. for 12 h with shaking at 200 rpm, resulting in a seed solution.

### 13.3 Fermentation Culture

The seed solution obtained in step 13.2 was inoculated at an inoculum volume of 5% into fermentation culture medium contained in a 5-L fermenter, resulting in a total volume of 3 L. After a fermentation pH was controlled to 7.0 with 25% (v/v) ammonia water, fermentation culture was started at a temperature of 50° C. under aeration at an aeration rate of 1.0 vvm and agitation at a speed of 300 rpm. After 6-8 hours, Bacillus licheniformis was fermented to an OD₆₀₀ of 6.5-8.0. At this time, the aeration was stopped, and the agitation speed was decreased to 80 rpm. The fermentation was then resumed and ended 60 h later. Subsequently, 15 mL microbial alanine racemase (in the form of centrifuged and resuspended Escherichia coli cells, which can produce alanine racemase by fermentation) was added, and the fermentation was resumed and run for 12 h at 50° C. and 80 rpm. During the fermentation process, when a concentration of glucose dropped to 30 g/L to 50 g/L, glucose was supplemented to increase the concentration to 100 g/L. After that, upon the glucose concentration dropping to 20 g/L to 40 g/L, glucose was again supplemented to increase the concentration to 70 g/L. FIG. 3 is a plot diagram of this fermentation process. After the fermentation process, a DL-alanine concentration in the fermentation broth was detected as 132.6 g/L, corresponding to a glucose-to-alanine conversion rate of 74.3%, and the optical purity of L-alanine was 49.6%.

### EXAMPLE 14: FERMENTATION CULTURE RESULTS OF BN11ΔLDH_{TI}-PFYAK STRAIN CONSTRUCTED IN EXAMPLE 2

Direct fermentation culture of the BN11Δldh_{Ti}-PFYAK strain obtained in Example 2 was carried out in accordance with the method and experimental parameters of Example 7.

As a result, a mixture of L- and D-alanine was produced at a yield of 64 g/L, and the optical purity of L-alanine was only 67.7% and therefore unsatisfactory in terms of racemism.

### EXAMPLE 15: FERMENTATION CULTURE RESULTS OF BA-1 STRAIN CONSTRUCTED IN EXAMPLE 3

Direct fermentation culture of the BA-1 strain obtained in Example 3 was carried out in accordance with the method and experimental parameters of Example 7.

As a result, a mixture of L- and D-alanine was produced at a yield of 129 g/L, and the optical purity of L-alanine was 70.8% and therefore unsatisfactory in terms of racemism.

The results of Examples 14 and 15 show that knockout of the alanine racemase genes of Bacillus licheniformis is necessary for racemic DL-alanine production by direct fermentation according to the present invention.

### EXAMPLE 16: KNOCKOUT OF ONE ALANINE RACEMASE GENE

Direct fermentation culture of a strain with either of the alr1 (SEQ ID NO. 29) and alr2 (SEQ ID NO. 30) genes encoding alanine racemase being knocked out using the method of Example 4 was carried out in accordance with the method of Example 7.

As a result, it was found to be unsatisfactory in terms of racemism.

Preferred specific embodiments of the present invention have been described in detail above. It is to be understood that, those of ordinary skill in the art can make various modifications and changes based on the concept of the present invention without exerting any creative effort. Accordingly, all the technical solutions that can be obtained by those skilled in the art by logical analysis, inference or limited experimentation in accordance with the concept of the present invention on the basis of the prior art are intended to fall within the protection scope as defined by the claims.

1. A method of constructing a DL-alanine-producing genetically engineered strain, comprising steps of:
providing an original strain possessing a pyruvate synthesis pathway;
constructing a genetically engineered strain for a two-step method by engineering a genome of the original strain through steps S200 and S300, or constructing a genetically engineered strain overexpressing an alanine racemase gene through steps S200, S300 and S400;
S200: inserting a copy of a 6-phosphofructokinase gene pfk and a copy of a pyruvate kinase gene pyk;
S300: inserting a gene GSald for alanine dehydrogenase thermostable at 42° C. to 55° C.;
S400: inactivating or deleting an alanine racemase gene and introducing an overexpressed alanine racemase gene.

2. The method of claim 1, wherein the original strain further possesses a lactate synthesis pathway and the genome of the original strain contains a lactate dehydrogenase gene;
the method further comprises a step of:
S100: inactivating or deleting a lactate dehydrogenase gene in the genome of the original strain.

3. The method of claim 2, wherein the original strain further possesses a D-lactate synthesis pathway and the genome of the original strain contains a D-lactate dehydrogenase gene ldh_{Ti};
the method further comprises a step of S500: inactivating or deleting a D-lactate dehydrogenase gene ldh_{Ti} in the genome of the original strain;
preferably, a sequence of the D-lactate dehydrogenase gene ldh_{Ti} is as shown in SEQ ID NO. 43.

4. The method of claim 1, wherein
a sequence of the 6-phosphofructokinase gene pfk is as shown in SEQ ID NO. 41, a sequence of the pyruvate kinase gene pyk is as shown in SEQ ID NO. 42; and/or a sequence of the gene for alanine dehydrogenase is as shown in SEQ ID NO. 1.

5. The method of claim 1, wherein in step S200 and step S300, relevant genes are inserted by adding their copies to a chromosome and ligating promoters in series upstream thereof.

6. The method of claim 5, wherein the promoter is Pₐₗₛ; preferably, a sequence of the promoter is as shown in SEQ ID NO. 4.

7. The method of claim 1, wherein
in step S400, an alanine racemase gene alr1 and an alanine racemase gene alr2 are completely inactivated or completely deleted; and/or
in step S400, the introduction of the overexpressed alanine racemase gene is accomplished by inserting a strong promoter and alanine racemase gene(s), and
inserted alanine racemase gene(s) is/are an alanine racemase gene alr1 or/and an alanine racemase gene alr2.

8. The method of claim 7, wherein
a sequence of the alanine racemase gene alr1 is as shown in SEQ ID NO. 2, a sequence of the alanine racemase gene alr2 is as shown in SEQ ID NO. 3; and/or
a sequence of the strong promoter is as shown in SEQ ID NO. 4.

9. The method of claim 2, wherein engineering of the genome of the original strain comprises step S100 and step S200.

10. The method of claim 2, wherein engineering of the genome of the original strain comprises step S100, step S200, step S300 and step S400.

11. The method of claim 10, comprising steps of:
S500: knocking out a D-lactate dehydrogenase gene ldh_{Ti} contained in the genome of the original strain;
S200: inserting a copy of the 6-phosphofructokinase gene pfk and a copy of the pyruvate kinase gene pyk;
S300: inserting a heterologous alanine dehydrogenase gene GSald; and
S400: knocking out an alanine racemase gene alr1 and an alanine racemase gene alr2 and then introducing an overexpressed alanine racemase gene alr1 or/and an overexpressed gene alanine racemase alr2.

12. The method of claim 1, wherein the DL-alanine-producing genetically engineered strain is capable of producing DL-alanine by fermentation at 42° C. to 55° C.

13. The method of claim 1, wherein the original strain is a thermophilic strain.

14. The method of claim 1, wherein the original strain is Bacillus.

15. The method of claim 14, wherein the original strain is Bacillus licheniformis, Bacillus coagulans, Bacillus methylotrophicus, thermophilic Bacillus inulinus or Geobacillus stearothermophilus.

16. The method of claim 14, wherein the original strain is Bacillus licheniformis ATCC 14580 or a derivative thereof.

17. The method of claim 14, wherein the original strain is Bacillus licheniformis BN11, deposited in the China Center for Type Culture Collection on Jan. 8, 2016 as CCTCC NO: M2016026.

18-28. (canceled)

29. The method of claim 2, wherein the DL-alanine-producing genetically engineered strain is capable of producing DL-alanine by fermentation at 42° C. to 55° C.

30. The method of claim 2, wherein the original strain is a thermophilic strain.

31. The method of claim 2, wherein the original strain is Bacillus.
